SEQUENCE LISTING (13 sequences)

SEQ ID NO: 1 - protein, 9 aa, Artificial sequence
  CDR1
GHFSNLAVN

SEQ ID NO: 2 - protein, 17 aa, Artificial sequence
  CDR2
GILWSGGSTFYADSVKG

SEQ ID NO: 3 - protein, 3 aa, Artificial sequence
  CDR3
GTN

SEQ ID NO: 4 - protein, 10 aa, Artificial sequence
  CDR1
GLTLSQYTMG

SEQ ID NO: 5 - protein, 17 aa, Artificial sequence
  CDR2
AIHWTSSVTDYADSVYG

SEQ ID NO: 6 - protein, 12 aa, Artificial sequence
  CDR3
THYYTHRGPFDY

SEQ ID NO: 7 - protein, 111 aa, Artificial sequence
  VHH1
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSS

SEQ ID NO: 8 - protein, 121 aa, Artificial sequence
  VHH2
QVQLVESGGGVVQPGGSLRLSCAASGLTLSQYTMGWFRQAPGKERELVAAIHWTSSVTDYADSVYGRFTISRDDSKNTGYLQMNSLRAEDTAVYYCAATHYYTHRGPFDYWGQGTLVTVSS

SEQ ID NO: 9 - DNA, 333 bp, Artificial sequence
  VHH1
gaggtgcagctggtggagtccggaggcggactggtgcagcctggaggaagcctgagactgagctgcgccgctagcggccacttcagcaacctggccgtgaactggttcaggcaggcccctggcaaggagagggagctggtggctggcatcctgtggagcggcggaagcaccttctacgccgacagcgtgaagggcaggttcaccatcagcaggggcaacgccgagaacatgctgtacctgcagatgaactccctgagggccgaggacaccgccgtgtactactgcaacaccggcaccaactggggccagggcacactcgtgaccgtgagcagc

SEQ ID NO: 10 - DNA, 363 bp, Artificial sequence
  VHH2
caagttcagctggtggaaagcggcggtggtgttgttcagccgggtggcagtctgcgtctgagctgcgcagccagtggtctgactttaagccagtataccatgggttggtttcgccaagctccgggtaaagaacgcgaactggtggccgccattcattggaccagcagcgtgaccgattatgccgatagcgtgtacggccgctttaccattagccgcgatgatagcaaaaatactggttatctgcagatgaattctttacgcgccgaagataccgccgtgtattactgcgccgccacccactactatacccatcgcggcccctttgattactggggtcaaggtactttagtgaccgtgagcagc

SEQ ID NO: 11 - protein, 230 aa, homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 12 - protein, 36 aa, Artificial sequence
  linker
GGGGGSGGGGSGGGGSGGGGSGGGGSGGGGSGGGGS

SEQ ID NO: 13 - protein, 498 aa, Artificial sequence
  Full length
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSQVQLVESGGGVVQPGGSLRLSCAASGLTLSQYTMGWFRQAPGKERELVAAIHWTSSVTDYADSVYGRFTISRDDSKNTGYLQMNSLRAEDTAVYYCAATHYYTHRGPFDYWGQGTLVTVSS

(19) United States
(12) Patent Application Publication
(10) Pub. No.: US 2022/0348664 A1
(43) Pub. Date: Nov. 3, 2022

(54) A NOVEL ANTI-PD-L1/ANTI-LAG-3 BISPECIFIC ANTIBODY AND USES THEREOF

(71) Applicant: WUXI BIOLOGICS IRELAND LIMITED, DUNDALK, CO LOUTH (IE)

(72) Inventors: Yunying CHEN, Shanghai (CN); Yi QIN, Shanghai (CN); Zhuozhi WANG, Shanghai (CN); Jing LI, Shanghai (CN)

(21) Appl. No.: 17/763,113

(22) PCT Filed: Sep. 25, 2020
(86) PCT No.: PCT/CN2020/117922

§ 371 Date: Mar. 23, 2022

(30) Foreign Application Priority Data
Sep. 26, 2019 (CN) PCT/CN2019/108062

(51) Int. Cl. C07K 16/28; A61P 35/00

(52) U.S. Cl. CPC C07K 16/2827; C07K 16/2803; A61P 35/00; A61K 2039/505

(57) ABSTRACT

Provided are bispecific antibodies against PD-L1 and LAG-3, the nucleic acid molecules encoding the antibodies, expression vectors and host cells used for the expression of the antibodies. Provided are the methods for validating the function of antibodies in vivo and in vitro. The antibody is a potent agent for the treatment of cancers via modulating immune functions.

### CROSS-REFERENCE TO RELATED APPLICATION

The application claims the benefit of International application PCT/CN2019/108062, filed Sep. 26, 2019, the entire contents of which are hereby incorporated by reference.

### SEQUENCE LISTING

The present application is filed with a Sequence Listing in electronic form. The entire contents of the Sequence Listing are hereby incorporated by reference.

### FIELD

This application generally relates to antibodies. More specifically, the application relates to bispecific antibodies against PD-L1 and LAG-3, a method for preparing the same, and the use of the antibodies.

### BACKGROUND

Over the last few years, immunotherapy has evolved into a very promising new frontier for fighting some types of cancers. PD-1, one of the immune-checkpoint proteins, is an inhibitory member of CD28 family expressed on activated CD4+ T cells and CD8+ T cells as well as on B cells. Its ligand PD-L1 is a type 1 transmembrane protein that has been speculated to play a major role in suppressing the adaptive arm of immune system. The binding of PD-L1 to PD-1 transmits an inhibitory signal based on interaction with phosphatases (SHP-1 or SHP-2) via Immunoreceptor Tyrosine-Based Switch Motif (ITSM). As a result, this pathway inhibits T cell proliferation and T cell functions such as cytokine production and cytotoxic activity. PD-1/PD-L1 axis plays a major role in down-regulating the immune system [1, 2].

Monoclonal antibodies targeting PD-1 or PD-L1 can block PD-1/PD-L1 binding and boost the immune response against cancer cells. These drugs have shown a great deal of promise in treating certain cancers. Multiple approved therapeutic antibodies targeting PD-1/PD-L1 have been developed by several pharmaceutical companies, including Pembrolizumab (Keytruda), Nivolumab (Opdivo), Cemiplimab (Libtayo), Atezolizumab (Tecentriq), Avelumab (Bavencio) and Durvalumab (Imfinzi). These drugs have been shown to be effective in treating various types of cancer, including melanoma of the skin, non-small cell lung cancer, kidney cancer, bladder cancer, head and neck cancers, and Hodgkin lymphoma. They are also being studied for use against many other types of cancer [3].

Lymphocyte-activation gene 3, also known as LAG-3, is a type I transmembrane protein that is a member of the immune-globulin superfamily (IgSF). LAG-3 is a cell surface molecule expressed on activated T cells, NK cells, B cells and plasmacytoid dendritic cells, but not on resting T cells. LAG-3 shares approximately 20% amino acid sequence homology with CD4, but binds to MHC class II with higher affinity, providing negative regulation of T cell receptor signaling [4].

Blockade of LAG-3 in vitro augments T cell proliferation and cytokine production, and LAG-3-deficient mice have a defect in the downregulation of T cell responses induced by the superantigen staphylococcal enterotoxin B, by peptides or by Sendai virus infection. LAG-3 is expressed on both activated natural Treg (nTreg) and induced CD4+FoxP3+ Treg (iTreg) cells, where expression levels are higher than that observed on activated effector CD4+ T cells. Blockade of LAG-3 on Treg cells abrogates Treg cell suppressor function whereas ectopic expression of LAG-3 in non-Treg CD4+ T cells confers suppressive activity. On the basis of the immunomodulatory role of LAG-3 on T cell function in chronic infection and cancer, the predicted mechanism of action for LAG-3-specific monoclonal antibodies is to inhibit the negative regulation of tumor-specific effector T cells [5]. LAG-3 has also been revealed to interfere with the activity of T cells, resulting in acquired resistance to PD1/PDL1 inhibitors [6]. Furthermore, dual blockade of the PD-1 pathway and LAG-3 has been shown in mice and human to be more effective for anti-tumor immunity than blocking either molecule alone [7]. Besides, it was clinically discovered that only a minority of cancer patients respond to anti-PD1/PDL1 immunotherapy.

Thus, there is a need for antibodies targeting PD1/PDL1 and LAG-3 that can overcome PD-1 resistance and produce more effective anti-tumor immunity. In the present disclosure, bispecific antibodies against PD-L1 and LAG-3 have been generated.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

The present disclosure provides bispecific antibodies against PD-L1 and LAG-3. It also provides the nucleic acid molecules encoding the anti-PD-L1/anti-LAG-3 antibodies, expression vectors and host cells used for the expression of bispecific antibodies. The present disclosure further provides the methods for preparing the anti-PD-L1/anti-LAG-3 antibodies, and validating their functions in vivo and in vitro. The bispecific antibodies of the present disclosure provide a very potent agent for preventing or treating diseases or conditions comprising proliferative disorders, immune disorders, or infections.

In some aspects, the present disclosure provides a bispecific antibody or the antigen-binding portion thereof, comprising a first antigen-binding site that specifically binds to PD-L1 and a second antigen-binding site that specifically binds to an antigen different from PD-L1. In some embodiments, the antigen different from PD-L1 is LAG-3.

In some aspects, the present disclosure provides a bispecific antibody or an antigen-binding portion thereof, comprising a first antigen binding domain and a second antigen binding domain, wherein:

the first antigen binding domain comprises: a CDRH1 comprising SEQ ID NO: 1; a CDRH2 comprising SEQ ID NO: 2; and a CDRH3 comprising SEQ ID NO: 3; and

the second antigen binding domain comprises: a CDRH1 comprising SEQ ID NO: 4; a CDRH2 comprising SEQ ID NO: 5; and a CDRH3 comprising SEQ ID NO: 6.

In certain embodiments, the first and/or second antigen binding domain is a single variable domain, a VHH, sdAb or a nanobody. The VHH may be derived from a camelid animal, comprising an alpaca or a llama. In certain embodiments, the VHH is a humanized VHH.

In certain embodiments, the first antigen binding domain comprises a first heavy chain variable region comprising the amino acid sequence of SEQ ID NO: 7 or a homologous sequence thereof having at least 80% sequence identity yet retaining specific binding affinity to PD-L1; and/or

the second antigen binding domain comprises a second heavy chain variable region comprising the amino acid sequence of SEQ ID NO: 8 or a homologous sequence thereof having at least 80% sequence identity yet retaining specific binding affinity to LAG-3.

In some specific embodiments, the first antigen binding domain comprises a first heavy chain variable region consisting of SEQ ID NO: 7 and the second antigen binding domain comprises a second heavy chain variable region consisting of SEQ ID NO: 8.

In certain embodiments, from N terminal to C terminal, the first antigen binding domain is operably linked to a constant region, and the constant region is operably linked to the second antigen binding domain. Alternatively, from N terminal to C terminal, the second antigen binding domain is operably linked to a constant region, and the constant region is operably linked to the first antigen binding domain.

In certain embodiments, the constant region is a human IgG constant region, such as a human IgG Fc region. In certain embodiments, the human IgG Fc region is a human IgG1 Fc region. Further, the human IgG1 Fc region may comprise a LALA mutation compared to wild-type human IgG1 Fc, specifically, mutations of L234A and L235A, according to EU numbering. In some embodiments, the Fc region may comprise or consist of SEQ ID NO: 11.

In certain embodiments, the constant region is operably linked to the first and/or second antigen binding domain via a linker. The linker may be a peptide sequence, such as comprising (G4S)n with n=1-10. In certain specific embodiments, the linker comprises or consists of SEQ ID NO: 12.

In some embodiments, from N terminal to C terminal, the bispecific antibody as disclosed herein is in the following format: 1^{st} domain-constant region-linker-2^{nd} domain or 2^{nd} domain-constant region-linker-1^{st} domain. Specifically, the full length of the antibody or the antigen-binding portion thereof comprises or consists of SEQ ID NO: 13.

In some embodiments, the bispecific antibody or the antigen-binding portion thereof as described above is a monoclonal antibody, preferably a humanized antibody.

In some embodiments, the first antigen binding domain can specifically bind to a PD-L1 antigen and the second antigen binding domain can specifically bind to a LAG-3 antigen. The PD-L1 and LAG-3 antigens can be derived from cynomolgus monkey, mouse or human, among others. The PD-L1 and LAG-3 antigens can be expressed as soluble proteins or expressed at the cell surface. Preferably, the PD-L1 and LAG-3 proteins are human PD-L1 and LAG-3 proteins. In some specific embodiments, the above-described antibodies can specifically bind to human PD-L1 and LAG-3 proteins simultaneously.

In some embodiments, the bispecific antibody or the antigen-binding portion thereof has one or more of the following properties:

(a) specifically binding to human PD-L1 and LAG-3 protein simultaneously with a high affinity, without cross-reactive binding to human PD-L2 or human CD4;

(b) specifically binding to cyno PD-L1 and/or mouse PD-L1 protein;

(c) specifically binding to cyno LAG-3 and/or mouse LAG-3 protein;

(d) capable of blocking PD-1 binding to PD-L1;

(e) capable of blocking LAG-3 binding to MHC-II and LAG-3 binding to FGL-1;

(f) capable of inducing a higher level of cytokine (e.g. IL-2) production compared to anti-PD-L1 or anti-LAG-3 monospecific antibodies, a combination thereof, and other bispecific antibodies targeting PD-L1 and LAG-3;

(g) providing good thermal stability and being stable in human serum; and

(h) providing significantly better anti-tumor effect compared to anti-PD-L1 or anti-LAG-3 monospecific antibodies, a combination thereof, and other bispecific antibodies targeting PD-L1 and LAG-3.

In some aspects, the present disclosure provides an isolated nucleic acid molecule, comprising a nucleic acid sequence encoding the bispecific antibody or the antigen-binding portion thereof as defined above.

In some embodiments, the isolated nucleic acid molecule comprises a nucleic acid sequence selected from the group consisting of:

(A) a nucleic acid sequence that encodes a heavy chain variable region as set forth in SEQ ID NO: 7;
(B) a nucleic acid sequence as set forth in SEQ ID NO: 9; or
(C) a nucleic acid sequence that hybridized under high stringency conditions to the complementary strand of the nucleic acid sequence of (A) or (B).

In some embodiments, the isolated nucleic acid molecule comprises a nucleic acid sequence selected from the group consisting of:

(A) a nucleic acid sequence that encodes a light chain variable region as set forth in SEQ ID NO: 8;
(B) a nucleic acid sequence as set forth in SEQ ID NO: 10; or
(C) a nucleic acid sequence that hybridized under high stringency conditions to the complementary strand of the nucleic acid sequence of (A) or (B).

In some aspects, the present disclosure provides a vector comprising the nucleic acid molecule as defined above.

In some aspects, the present disclosure provides a host cell comprising the nucleic acid molecule or the vector as defined above.

In some aspects, the present disclosure provides a pharmaceutical composition comprising the bispecific antibody or the antigen-binding portion thereof as defined above and a pharmaceutically acceptable carrier.

In some aspects, the present disclosure provides a method for producing the bispecific antibody or the antigen-binding portion thereof as defined above, comprising the steps of:

expressing the bispecific antibody or antigen-binding portion thereof in the host cell as defined above; and

isolating the bispecific antibody or antigen-binding portion thereof from the host cell.

In some aspects, the present disclosure provides a method for modulating an immune response in a subject, comprising administering to the subject an effective amount of the bispecific antibody or the antigen-binding portion thereof or the pharmaceutical composition as defined herein to the subject, optionally the immune response is PD-L1 and/or LAG-3 related.

In some aspects, the present disclosure provides a method for inhibiting growth of tumor cells in a subject, comprising administering an effective amount of the bispecific antibody or the antigen-binding portion thereof or the pharmaceutical composition as defined herein to the subject.

In some aspects, the present disclosure provides a method for preventing or treating a disease or condition in a subject, comprising administering an effective amount of the bispecific antibody or the antigen-binding portion thereof or the pharmaceutical composition as defined herein to the subject, wherein the disease or condition is selected from a proliferative disorder, an immune disorder and an infectious disease. In some embodiments, the disease or condition is PD-L1 and/or LAG-3 related. In some embodiments, the proliferative disorder is cancer, such as colon cancer, lymphoma, lung cancer, liver cancer, cervical cancer, breast cancer, ovarian cancer, pancreatic cancer, melanoma, glioblastoma, prostate cancer, esophageal cancer, or gastric cancer. In some embodiments, the cancer is a colon cancer. In some embodiments, the disease or condition is a chronic infection.

In some embodiments, the bispecific antibody or antigen-binding portion thereof as defined herein may be administered in combination with a chemotherapeutic agent, radiation and/or other agents for use in cancer immunotherapy.

In some aspects, the present disclosure provides the bispecific antibody or the antigen-binding portion thereof for use

i) in the modulation of immune responses, such as restoring T cell activity;

ii) in enhancing T cell proliferation and cytokine production, such as IL-2 production; and/or

iii) in stimulating an immune response or function, such as boosting the immune response against cancer cells.

In some aspects, the present disclosure provides the bispecific antibody or the antigen-binding portion thereof as defined herein for use in treating or preventing proliferative disorders (such as cancers), immune disorders, or infections.

In some aspects, the present disclosure provides the bispecific antibody or the antigen-binding portion thereof as defined herein for use in diagnosing proliferative disorders (such as cancers), immune disorders, or infections.

In some aspects, the present disclosure provides use of the bispecific antibody or the antigen-binding portion thereof of as defined herein in the manufacture of a medicament for modulating an immune response or inhibiting growth of tumor cells in a subject.

In some aspects, the present disclosure provides use of the bispecific antibody or the antigen-binding portion thereof of as defined herein in the manufacture of a medicament for treating or preventing proliferative disorders (such as cancers), immune disorders, or infections.

In some aspects, the present disclosure provides a kit for treating or diagnosing proliferative disorders (such as cancers), immune disorders or infections, comprising a container comprising the bispecific antibody or the antigen-binding portion thereof as defined herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1A is a schematic graph of the W3669 bispecific antibody of the present disclosure and

FIG. 1B shows the specific sequence of the antibody.

FIG. 2 illustrates the result of SDS-PAGE (A) and SEC-HPLC (B) after Protein A chromatography.

FIG. 3 illustrates the binding of the antibodies to human PD-L1-expressed CHO-K1 cells, as measured by FACS.

FIG. 4 illustrates the binding of the antibodies to human PD-L1 protein, as measured by ELISA.

FIG. 5 illustrates the binding of the antibodies to human LAG-3-expressing 293 cells, as measured by FACS.

FIG. 6 illustrates the binding of the antibodies to recombinant human LAG-3 protein, as measured by ELISA.

FIG. 7 illustrates the dual binding of the antibodies to both human PD-L1 and LAG-3 protein, as measured by ELISA.

FIG. 8 illustrates the binding of the antibodies to human PD-L1 expressing cells and LAG-3 expressing cells, as measured by FACS.

FIG. 9 illustrates the cross-reactivity of the W3669 bispecific antibody to human PD-L2 (A) and human CD4 (B), as measured by FACS and ELISA, respectively. The positive control is a commercial anti-CD4 antibody diluted in 1:100.

FIG. 10 illustrates the binding of the antibodies to recombinant cyno PD-L1 protein, as measured by ELISA.

FIG. 11 illustrates the binding of the antibodies to recombinant mouse PD-L1 protein, as measured by ELISA.

FIG. 12 illustrates the binding of the antibodies to recombinant cyno LAG-3 protein, as measured by ELISA.

FIG. 13 illustrates the binding of the antibodies to recombinant mouse LAG-3 protein, as measured by ELISA.

FIG. 14 illustrates the blockade of PD-1 binding to PD-L1 expressing cells, as measured by FACS.

FIG. 15 illustrates the blockade of LAG-3 binding to MHC-II expressing Raji cells, as measured by FACS.

FIG. 16 illustrates the blockade of FGL-1/LAG-3 Interaction, as measured by ELISA.

FIG. 17 illustrates the result of PD-1 reporter gene assay.

FIG. 18 illustrates the result of LAG-3 reporter gene assay.

FIG. 19 illustrates the result of dual Pathway Reporter Gene Assay.

FIG. 20 illustrates IL-2 production in human allogeneic mixed lymphocyte reaction (MLR).

FIG. 21 illustrates the secreted IL-2 level (A) and fold change (B) in human PBMCs activated by SEB. The PD-L1 mab, LAG-3 mab and “combo” are parental anti-PD-L1 VHH, parental anti-LAG-3 VHH and anti-PD-L1 VHH+anti-LAG-3 VHH, respectively.

FIG. 22A-C show the thermal stability measured by differential scanning fluorimetry (DSF). FIG. 22A is a table summarizing the result, FIGS. 22B and 22C are the melting curve plot corresponding to W3669 antibody and W366-BMK1, respectively. The higher Tm1 of the W3669 bispecific antibody indicated better thermal stability than the bispecific W366-BMK1.

FIG. 23 illustrates the result of serum stability test.

FIG. 24 illustrates the anti-tumor effect of different antibodies in Colon 26 syngeneic model. The arrows indicate the time points of administration. BsAb refers to the W3669 antibody. The PD-L1 mab and LAG-3 mab are W315-BMK8 and parental anti-LAG-3 VHH that both have mouse cross reactivity, respectively.

FIG. 25 illustrates the dose response of anti-tumor effect in Colon 26 syngeneic model. The arrows indicate the time points of administration.

FIG. 26A illustrates the pharmacokinetics profile in mouse. The result indicated that the W3669 antibody has normal PK profile in mouse at high dose.

FIG. 26B illustrates the result of anti-drug antibody generation in mouse.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

All publications, patent applications, patents, and other references mentioned herein are incorporated by reference in their entirety.

### Definitions

In order to better understand the disclosure, the definitions and explanations of the relevant terms are provided as follows.

The term “antibody” or “Ab,” herein is used in the broadest sense, which encompasses various antibody structures, including polyclonal antibodies, monospecific and multispecific antibodies (e.g. bispecific antibodies). The term antibody generally refers to a Y-shaped tetrameric protein comprising two heavy (H) and two light (L) polypeptide chains held together by covalent disulfide bonds and non-covalent interactions. Light chains of an antibody may be classified into κ and λ, light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Chothia et al., (1989) Nature 342:878-883. Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

The term “antigen-binding domain” (e.g. LAG-3 binding domain or PD-L1 binding domain) as used herein refers to an antibody fragment formed from a portion of an antibody comprising one or more CDRs, or any other antibody fragment that binds to an antigen but does not comprise an intact native antibody structure. Examples of antigen-binding domain include, without limitation, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding domain is capable of binding to the same antigen to which the parent antibody binds. In certain embodiments, the antigen-binding domain is a VHH domain. In some embodiments, the antigen-binding domain may comprise one or more CDRs from a particular camelid VHH domain grafted to a framework region from one or more different human antibodies. For more and detailed formats of antigen-binding domain are described in Spiess et al, Molecular Immunology, 67(2), pp. 95-106 (2015), and Brinkman et al., mAbs, 9(2), pp. 182-212 (2017), which are incorporated herein by reference.

The terms “single variable domain” or “VHH domain” may be used interchangeably herein and refers to a single chain antigen binding domain that is capable of binding to an antigen or epitope independently of a different variable domain. The VHH domain (i.e. variable domain of a heavy chain antibody) represents the smallest known antigen-binding unit generated by adaptive immune responses (Koch-Nolte F. et al., FASEB J. November; 21(13):3490-8. Epub 2007 Jun. 15 (2007)). The VHH domain may be a human domain, but also includes a single domain from other species such as rodent, nurse shark and Camelid VHH domains. Camelid VHH are immunoglobulin single variable domain polypeptides that are derived from species including camel, llama, alpaca, dromedary, and guanaco, which produce heavy chain antibodies naturally devoid of light chains. Such VHH domains may be humanized according to standard techniques available in the art, and such domains are considered to be “single domain antibodies”. As used herein VHH includes camelid VHH domains and humanized VHH domains.

The term “PD-L1”, also known as programmed death-ligand 1, is a 40 kDa type 1 transmembrane protein that has been speculated to play a major role in suppressing the adaptive arm of immune system. PD-L1 is the principal ligand of programmed death 1 (PD-1), a coinhibitory receptor that can be constitutively expressed or induced in myeloid, lymphoid, normal epithelial cells and in cancer. PD-L1 is expressed in placenta, spleen, lymph nodes, thymus, heart, fetal liver, and is also found on many tumor or cancer cells. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response.

The term “PD-L1” as used herein, when referring to the amino acid sequence of PD-L1 protein, including full-length PD-L1 protein, or the extracellular domain of PD-L1 (PD-L1 ECD) or fragment containing PD-L1 ECD; Fusion protein of PD-L1 ECD, for example, fragment fused with IgG Fc from mice or human (mFc or hFc) is also included. Moreover, as understood by a person skilled in the art, PD-L1 protein would also include those into which mutations of amino acid sequence are naturally or artificially introduced (including but not limited to replacement, deletion and/or addition) without affecting the biological functions. Therefore, in the present disclosure, the term “PD-L1 protein” should include all such sequences, including the sequence list above and its natural or artificial variants. In addition, when the sequence fragment of PD-L1 protein is referred, it means not only the above sequence fragment, but also the corresponding sequence fragment of natural or artificial variants.

As used herein, the term “cell surface-expressed PD-L1,” as used herein, refers to one or more PD-L1 protein(s) that is/are expressed on the surface of a cell in vitro or in vivo, such that at least a portion of a PD-L1 protein is exposed to the extracellular side of the cell membrane and is accessible to an antigen-binding portion of an antibody. A “cell surface-expressed PD-L1” can comprise or consist of a PD-L1 protein expressed on the surface of a cell which normally expresses PD-L1 protein. Alternatively, “cell surface-expressed PD-L1” can comprise or consist of PD-L1 protein expressed on the surface of a cell that normally does not express human PD-L1 on its surface but has been artificially engineered to express PD-L1 on its surface.

The term “an antibody that binds PD-L1” or an “anti-PD-L1 antibody” as used herein includes antibodies and antigen-binding fragments thereof that specifically recognize PD-L1. The antibodies and antigen-binding fragments of the present disclosure may bind soluble PD-L1 protein and/or cell surface expressed PD-L1. Soluble PD-L1 includes natural PD-L1 proteins as well as recombinant PD-L1 protein variants that lack a transmembrane domain or are otherwise unassociated with a cell membrane. The expression “anti-PD-L1 antibody” herein includes both monovalent antibodies with a single specificity, as well as bispecific antibodies comprising a first antigen-binding domain that binds PD-L1 and a second antigen-binding domain that binds a second (target) antigen, wherein the anti-PD-L1 antigen-binding domain comprises any of the HCVR/LCVR or CDR sequences as set forth in Table A herein. Examples of anti-PD-L1 bispecific antibodies are described elsewhere herein.

The term “PD-L2”, as used herein, refers to programmed cell death ligand 2, which competes with PD-L1 for binding to PD-1. A representative amino acid sequence of human PD-L2 is disclosed under the NCBI accession number: NP_079515.2.

The term “LAG-3” or “LAG-3”, also known as Lymphocyte-activation gene 3, is a type I transmembrane protein that is a member of the immune-globulin superfamily (IgSF). LAG-3 was designated CD223 (cluster of differentiation 223). LAG-3 is a cell surface molecule expressed on activated T cells, NK cells, B cells and plasmacytoid dendritic cells etc., but not on resting T cells. LAG-3 is an immune checkpoint receptor with diverse biologic effects on T cell function. As used herein, the term “LAG-3” includes variants, isoforms, homologs, orthologs and paralogs.

The term “human LAG-3”, as used herein, refers to a human-derived LAG-3 protein, such as having the complete amino acid sequence of human LAG-3 (Genbank Accession No. NP_002277). The human LAG-3 sequence may differ from human LAG-3 of Genbank Accession No. NP_002277 by having, e.g., conserved mutations or mutations in non-conserved regions and the LAG-3 has substantially the same biological function as the human LAG-3 of Genbank Accession No. NP_002277. For example, a biological function of human LAG-3 is having an epitope in the extracellular domain of LAG-3 that is specifically bound by an antibody of the instant disclosure or a biological function of human LAG-3 is binding to MHC Class II molecules or FGL1 like molecules.

The term “mouse LAG-3”, as used herein, refers to mouse derived LAG-3 protein, such as having the complete amino acid sequence of mouse LAG-3 (Genbank Accession No. NP_032505).

The term “cynomolgus LAG-3”, as used herein, refers to cynomolgus derived LAG-3 protein, such as having the complete amino acid sequence of cynomolgus LAG-3 (Genbank Accession No. XP_005570011.1).

The term “anti-LAG-3 antibody,” as used herein, refers to an antibody that specifically binds to LAG-3. An “anti-LAG-3 antibody” may include monovalent antibodies with a single specificity. Exemplary anti-LAG-3 antibodies are described elsewhere herein.

The term “bivalent,” as used herein refers to an antibody or an antigen-binding fragment having two antigen-binding sites; the term “monovalent” refers to an antibody or an antigen-binding fragment having only one single antigen-binding site; and the term “multivalent” refers to an antibody or an antigen-binding fragment having multiple antigen-binding sites. In some embodiments, the bispecific antibody or antigen-binding fragment thereof as disclosed herein is bivalent or tetravalent.

As used herein, a “bispecific” molecule refers to an artificial molecule which has fragments derived from two different monoclonal antibodies and is capable of binding to two different epitopes. The two epitopes may present on the same antigen, or they may present on two different antigens.

The term “bispecific antibody” or “bispecific antigen-binding molecule”, as used herein, means a protein, polypeptide or molecular complex comprising at least a first antigen-binding domain (i.e. PD-L1 binding domain) and a second antigen-binding domain (i.e. LAG-3 binding domain). Each antigen-binding domain within the bispecific antibody comprises at least one CDR that alone, or in combination with one or more additional CDRs and/or FRs, specifically binds to a particular antigen. In the context of the present disclosure, the first antigen-binding site specifically binds to a first antigen (e.g., PD-L1), and the second antigen-binding site specifically binds to a second, distinct antigen (e.g., LAG-3).

The term “Fc” with regard to an antibody refers to that portion of the antibody comprising the second and third constant regions of a first heavy chain bound to the second and third constant regions of a second heavy chain via disulfide bonding, optionally the Fc region also comprises a part or whole of the hinge region. The Fc portion of the antibody is responsible for various effector functions such as antibody-dependent cell-mediated cytotoxicity (ADCC), and complement dependent cytotoxicity (CDC), but does not function in antigen binding. Fc region herein includes both wild-type Fc regions and variants thereof having different mutations for various purposes. As used herein, the Fc may be a wild-type Fc region such as wild-type human IgG1 Fc region or a variant thereof.

The term “operably link” or “operably linked” refers to a juxtaposition, with or without a spacer or a linker or an intervening sequence, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antigen binding domain may be operably linked to a constant region so as to provide for a stable product with antigen-binding activity. For another example, an antigen-binding domain can be operably linked to another antigen-binding domain with an intervening sequence there between, and such intervening sequence can be a linker or can comprise a much longer sequence such as a constant region of an antibody. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “anti-PD-L1/anti-LAG-3 antibody”, “anti-PD-L1/anti-LAG-3 bispecific antibody”, “antibody against PD-L1 and LAG-3”, “anti-PD-L1×LAG-3 bispecific antibody”, “PD-L1×LAG-3 antibody”, as used herein interchangeably, refers to a bispecific antibody that specifically binds to PD-L1 and LAG-3.

The term “monoclonal antibody” or “mAb”, as used herein, refer to a preparation of antibody molecules of single molecular composition. A monoclonal antibody displays a single binding specificity and affinity for a particular epitope.

The term “humanized antibody” is intended to refer to antibodies in which CDR sequences derived from the germline of another mammalian species, such as a mouse, llama or alpaca, have been grafted onto human framework sequences. Additional framework region modifications may be made within the human framework sequences.

The term “recombinant antibody,” as used herein, refers to an antibody that is prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “Ka,” as used herein, is intended to refer to the association rate of a particular antibody-antigen interaction, whereas the term “Kd” as used herein, is intended to refer to the dissociation rate of a particular antibody-antigen interaction. Ka and Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁷ M or less, more preferably 5×10⁻⁸ M or less, more preferably 1×10⁻⁸ M or less, more preferably 5×10⁻⁹ M or less, more preferably 1×10⁻⁹ M, more preferably 8×10⁻¹⁰ M, more preferably 7×10¹⁰ M, more preferably 6×10⁻¹⁰ M, more preferably 5×10⁴⁰ M or less and even more preferably 4×10⁻¹⁰ M or less for a target antigen, as determined by SPR.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”. In some embodiments, the antibody as disclosed herein binds to human PD-L1 expressing cells with an EC₅₀ of no more than 1 nM, no more than 0.8 nM, no more than 0.5 nM, no more than 0.4 nM, preferably no more than 0.3 nM, more preferably about 0.2 nM, as determined by FACS. In some embodiments, the antibody as disclosed herein binds to human PD-L1 protein with an EC₅₀ of no more than 1 nM, no more than 0.1 nM, no more than 0.05 nM, no more than 0.04 nM or preferably no more than 0.03 nM, as determined by ELISA.

The ability of “block binding” or “inhibit binding,” as used herein, refers to the ability of an antibody or antigen-binding fragment thereof to block or inhibit the binding of two molecules (eg, PD1 binding to PD-L1, LAG-3 binding to MHC-II, or LAG-3 binding to FGL-1) to any detectable level. In certain embodiments, the binding of the two molecules can be inhibited at least 50% by the antibody or antigen-binding fragment thereof. In certain embodiments, such an inhibitory effect may be greater than 60%, greater than 70%, greater than 80%, or greater than 90%. In some embodiments, the binding of PD1 to its ligand PD-L1 (e.g. on PD-L1 expressing cells) can be inhibited by the antibody or antigen-binding fragment thereof with an IC₅₀ (i.e. 50% inhibiting concentration) of no more than 1 nM, no more than 0.5 nM, no more than 0.4 nM, no more than 0.3 nM or preferably no more than 0.2 nM, as determined by FACS.

The term “epitope,” as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996). In a linear epitope, all the interaction sites between a protein and an interaction molecule (e.g., an antibody) are present linearly along the primary amino acid sequence of the protein. In a conformational epitope, the interaction sites span over amino acid residues that are separate from each other in a protein. Antibodies may be screened depending on competitiveness of binding to the same epitope by conventional techniques known by a person skilled in the art. For example, study on competition or cross-competition may be conducted to obtain antibodies that compete or cross-compete with each other for binding to antigens (e.g. RSV fusion protein). High-throughput methods for obtaining antibodies binding to the same epitope, which are based on their cross-competition, are described in an international patent application WO 03/48731.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds a PD-L1/LAG-3 protein is substantially free of antibodies that specifically bind antigens other than PD-L1/LAG-3 proteins). An isolated antibody that specifically binds a human PD-L1/LAG-3 protein may, however, have cross-reactivity to other antigens, such as PD-L1/LAG-3 proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In some embodiments of the disclosure, human PD-L1/LAG-3 gene was transfected into 293F cells.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example section 3.5 and Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017 Nov. 9). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “cancer,” as used herein, refers to or describe the physiological condition in mammals that is typically characterized by unregulated cell growth. It may refer to any of a tumor or a malignant cell growth, proliferation or metastasis-mediated, and may be solid tumors and non-solid tumors such as leukemia.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For instance, the “an effective amount,” when used in connection with treatment of PD-L1/LAG-3-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

Bispecific Antibodies and Antigen-Binding Portion Thereof

In certain embodiments, the antibodies and antigen-binding fragments thereof provided herein are bispecific. In some embodiments, the bispecific antibodies and antigen-binding fragments thereof provided herein has a first specificity for PD-L1, and a second specificity different from PD-L1. In some embodiments, the second specificity is for a second antigen different from PD-L1 and optionally, whose blockade may produce a synergetic effect than blocking one antigen alone. Specifically, the second antigen may be LAG-3.

According to certain exemplary embodiments, the present disclosure includes a bispecific antibody or the antigen-binding portion thereof, comprising a first antigen-binding domain that specifically binds to PD-L1 and a second antigen-binding domain that specifically binds to LAG-3. Such antibodies may be referred to herein as, e.g., “anti-PD-L1/anti-LAG-3,” or “anti-PD-L1/LAG-3,” or “anti-PD-L1×LAG-3” or “PD-L1×LAG-3” bispecific antibodies, or other similar terminology.

The bispecific antibody of the disclosure is capable of binding to PD-L1 or LAG-3 antigen with high affinity. The PD-L1 and LAG-3 antigens, as disclosed herein, can be derived from cynomolgus monkey, mouse and human, among others. The PD-L1 and LAG-3 antigens can be expressed as soluble proteins or expressed at the cell surface. Preferably, the PD-L1 and LAG-3 antigens are human PD-L1 and LAG-3 proteins.

The binding of an antibody of the disclosure to PD-L1 or LAG-3 can be assessed using one or more techniques well established in the art, for instance, ELISA. The binding specificity of an antibody of the disclosure can also be determined by monitoring binding of the antibody to cells expressing a PD-L1 protein or LAG-3 protein, e.g., flow cytometry. For example, an antibody can be tested by a flow cytometry assay in which the antibody is reacted with a cell line that expresses human PD-L1, such as CHO cells that have been transfected to express PD-L1 on their cell surface. Additionally or alternatively, the binding of the antibody, including the binding kinetics (e.g., K_{D} value) can be tested in BIAcore binding assays. Still other suitable binding assays include ELISA or FACS assays, for example using a recombinant PD-L1 protein. For instance, an antibody of the disclosure binds to a human PD-L1 protein with a K_{D} of 1×10⁻⁷ M or less, binds to a human PD-L1 protein with a K_{D} of 5×10⁻⁸ M or less, binds to a human PD-L1 protein with a K_{D} of 2×10⁻⁸ M or less, binds to a human PD-L1 protein with a K_{D} of 1×10⁻⁸ M or less, binds to a human PD-L1 protein with a K_{D} of 5×10⁻⁹ M or less, binds to a human PD-L1 protein with a K_{D} of 4×10⁻⁹ M or less, binds to a human PD-L1 protein with a K_{D} of 3×10⁻⁹ M or less, binds to a human PD-L1 protein with a K_{D} of 2×10⁻⁹ M or less, binds to a human PD-L1 protein with a K_{D} of 1×10⁻⁹ M or less, binds to a human PD-L1 protein with a K_{D} of 5×10⁻¹° M or less, or binds to a human PD-L1 protein with a K_{D} of 4×10⁻¹⁰ M or less, as measured by Surface Plasmon Resonance.

For example, an antibody can be tested by a flow cytometry assay in which the antibody is reacted with a cell line that expresses human LAG-3, such as CHO or 293F cells that have been transfected to express LAG-3 on their cell surface. Additionally or alternatively, the binding of the antibody, including the binding kinetics (e.g., K_{D} value) can be tested in BIAcore binding assays. Still other suitable binding assays include ELISA or FACS assays, for example using a recombinant LAG-3 protein. For instance, an antibody of the disclosure binds to a human LAG-3 protein with a K_{D} of 1×10⁻⁷ M or less, binds to a human LAG-3 protein with a K_{D} of 5×10⁻⁸ M or less, binds to a human LAG-3 protein with a K_{D} of 2×10⁻⁸ M or less, binds to a human LAG-3 protein with a K_{D} of 1×10⁻⁸ M or less, binds to a human LAG-3 protein with a K_{D} of 5×10⁻⁹ M or less, binds to a human LAG-3 protein with a K_{D} of 4×10⁻⁹ M or less, binds to a human LAG-3 protein with a K_{D} of 3×10⁻⁹ M or less, binds to a human LAG-3 protein with a K_{D} of 2×10⁻⁹ M or less, binds to a human LAG-3 protein with a K_{D} of 1×10⁻⁹ M or less, or binds to a human LAG-3 protein with a K_{D} of 5×10⁻¹⁰ M or less, as measured by Surface Plasmon Resonance.

The First Antigen Binding Domain that Specifically Binds to PD-L1

The PD-L1 binding domain as disclosed herein may be selected from a variety of antibody forms or fragments that can specifically bind to PD-L1. In some embodiments, the PD-L1 binding domain may be, such as but not limited to, Fab, F(ab′)2, scFv, VHH, and dAb. Specifically, the PD-L1 binding domain is a VHH domain. In some embodiments, the PD-L1 binding domain comprises or consists of a heavy chain variable region or domain. Specifically, the heavy chain variable region or domain may comprise one or more heavy chain CDRs (CDRHs) selected from the group consisting of:

(i) a CDRH1 comprising SEQ ID NO: 1 or an amino acid sequence that differs from SEQ ID NO: 1 by an amino acid addition, deletion or substitution of not more than 2 amino acids;

(ii) a CDRH2 comprising SEQ ID NO: 2 or an amino acid sequence that differs from SEQ ID NO: 2 by an amino acid addition, deletion or substitution of not more than 2 amino acids; and

(iii) a CDRH3 comprising SEQ ID NO: 3 or an amino acid sequence that differs from SEQ ID NO: 3 by an amino acid addition, deletion or substitution of not more than 2 amino acids.

In some specific embodiments, the heavy chain variable region or domain comprises (i) a CDRH1 comprising or consisting of SEQ ID NO: 1; (ii) a CDRH2 comprising or consisting of SEQ ID NO: 2; and (iii) a CDRH3 comprising or consisting of SEQ ID NO: 3.

In some embodiments, the heavy chain variable region of PD-L1 binding domain comprises: (i) the amino acid sequence of SEQ ID NO: 7; (ii) an amino acid sequence at least 85%, 90%, or 95% identical to SEQ ID NO: 7; or (iii) an amino acid sequence with addition, deletion and/or substitution of one or more amino acids compared with SEQ ID NO: 7.

The PD-L1 binding domain may be a heavy chain variable domain, which is interchangeably used herein with the terms “VHH”, “VHH domain”, “V_{HH}” or “Nanobody,” etc. V_{HH} molecules derived from Camelidae antibodies are among the smallest intact antigen-binding domains known (approximately 15 kDa, or 10 times smaller than a conventional IgG) and hence are well suited towards delivery to dense tissues and for accessing the limited space between macromolecules.

Single variable domains or VHHs may be made by the skilled artisan according to methods known in the art or any future method. For example, VHHs may be obtained using methods known in the art such as by immunizing a camel and obtaining hybridoma's therefrom, or by cloning a library of VHHs using molecular biology techniques known in the art and subsequent selection by using phage display.

For instance, a VHH can be obtained by immunization of llamas or alpacas with the desired antigen and subsequent isolation of the mRNA coding for heavy-chain antibodies. By reverse transcription and polymerase chain reaction, a gene library of single-domain antibodies containing several million clones is produced. Screening techniques like phage display and ribosome display help to identify the clones binding the antigen. One technique is phage display in which a library of (e.g., human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., Barbas et al., Proc. Natl. Acad. Sci. USA 88:7978-7982 (1991)).

When the most potent clones have been identified, their DNA sequence is optimized, for example, by affinity maturation or humanization. Humanization may prevent immunological reactions of the human organism against the antibody.

Accordingly, VHH domains can be obtained (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, in particular a species of mammal, such as from a human being, or by expression of a nucleic acid encoding such a camelized VH domain; (5) by “camelisation” of a “domain antibody” or “dAb”, or by expression of a nucleic acid encoding such a camelized VH domain; (6) using synthetic or semi-synthetic techniques for preparing proteins, polypeptides or other amino acid sequences; (7) by preparing a nucleic acid encoding a

VHH using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

Single variable domains or single-domain antibodies (sdAb) are usually generated by PCR cloning of variable domain repertoire from blood, lymph node, or spleen cDNA obtained from immunized animals into a phage display vector. Antigen-specific single-domain antibodies are commonly selected by panning phage libraries on immobilized antigen, e.g., antigen coated onto the plastic surface of a test tube, biotinylated antigens immobilized on Streptavidin beads, or membrane proteins expressed on the surface of cells. The affinity of sdAbs can often been improved by mimicking this strategy in vitro, for instance, by site directed mutagenesis of the CDR regions and further rounds of panning on immobilized antigen under conditions of increased stringency (higher temperature, high or low salt concentration, high or low pH, and low antigen concentrations) (Wesolowski et al., Single domain antibodies: promising experimental and therapeutic tools in infection and immunity. Med Microbiol Immunol (2009) 198: 157-174).

Methods for preparing a VHH specifically binding to an antigen or epitope was described in references, for example: R. van der Linden et al., Journal of Immunological Methods, 240(2000) 185-195; Li et al., J Biol Chem., 287(2012) 13713-13721; Deffar et al., African Journal of Biotechnology Vol. 8(12), pp. 2645, 17 Jun. 2009 and WO94/04678.

In some embodiments, the first VHH in the bispecific antibodies is fused to an Fc-domain of an antibody, for example, Fc-domain of IgG (e.g., IgG4 or IgG1). In some specific embodiments, the Fc-domain is an Fc-domain of human IgG1. By fusing a VHH to a Fc domain, it may be more efficient to recruit effector functions. Also, the fusion of VHH to Fc domain may help the polypeptide chain to form a dimer, and may also help the extension of the half life of the bispecific antibodies in vivo.

The Second Antigen Binding Domain that Specifically Binds to LAG-3

Similarly, the LAG-3 binding domain as disclosed herein may be selected from a variety of antibody forms or fragments that can specifically bind to LAG-3. In some embodiments, the LAG-3 binding domain may be, such as but not limited to, Fab, F(ab′)2, scFv, VHH, and dAb. Preferably, the LAG-3 binding domain is a VHH domain. In some embodiments, the second antigen-binding domain comprises or consists of a heavy chain variable region or domain, which comprises one or more heavy chain CDRs (CDRHs) selected from the group consisting of:

(i) a CDRH1 comprising SEQ ID NO: 4 or an amino acid sequence that differs from SEQ ID NO: 4 by an amino acid addition, deletion or substitution of not more than 2 amino acids;

(ii) a CDRH2 comprising SEQ ID NO: 5 or an amino acid sequence that differs from SEQ ID NO: 5 by an amino acid addition, deletion or substitution of not more than 2 amino acids; and

(iii) a CDRH3 comprising SEQ ID NO: 6 or an amino acid sequence that differs from SEQ ID NO: 6 by an amino acid addition, deletion or substitution of not more than 2 amino acids.

In some embodiments, the heavy chain variable region or domain comprises (i) a CDRH1 comprising or consisting of SEQ ID NO: 4; (ii) a CDRH2 comprising or consisting of SEQ ID NO: 5; and (iii) a CDRH3 comprising or consisting of SEQ ID NO: 6.

In some embodiments, the heavy chain variable region of the second single variable domain comprises: (i) the amino acid sequence of SEQ ID NO: 8; (ii) an amino acid sequence at least 85%, 90%, or 95% identical to SEQ ID NO: 8; or (iii) an amino acid sequence with addition, deletion and/or substitution of one or more amino acids compared with SEQ ID NO: 8.

The assignment of amino acids to each CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted.

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In some embodiments, the amino acid sequences of the variable region can be at least 90%, 91%, 92%, 93%, 94%, 95%, 96%, 97%, 98% or 99% identical to the respective sequences set forth above.

Preferably, the CDRs of the isolated antibody or the antigen-binding portion thereof contain a conservative substitution of not more than 2 amino acids, or not more than 1 amino acid. The term “conservative substitution”, as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

Generation of Bispecific Antibodies

The bispecific antibodies and antigen-binding portions provided herein can be made with any suitable methods known in the art. In a conventional approach, two heavy chain single variable domains having different antigenic specificities can be co-expressed in a host cell to produce bispecific antibodies in a recombinant way, followed by purification by e.g. affinity chromatography.

Recombinant approach may also be used, where sequences encoding the antibody heavy chain variable domains for the two specificities are respectively fused to immunoglobulin constant domain sequences, followed by insertion to an expression vector and transfected to a suitable host cell for recombinant expression of the bispecific antibody.

In certain embodiments, the first antigen-binding domain and the second antigen-binding domain of the bispecific antibody may be directly or indirectly connected to one another. In certain embodiments, the first antigen-binding domain and the second antigen-binding domain of the bispecific antibody may be connected to one another by a linker. In a specific embodiment, the linker is a peptide linker. In certain embodiments, the first antigen-binding domain and the second antigen-binding domain of the bispecific antibody may be connected to one another by a sequence comprising a linker and a Fc region.

In certain embodiments, the first antigen-binding domain and the second antigen-binding domain of the bispecific antibody may be directly or indirectly connected to one another and further bound to an Fc region to form a bispecific antigen-binding molecule of the present disclosure. Alternatively, the first antigen-binding domain and the second antigen-binding domain may be connected to an Fc region. Bispecific antigen-binding molecules of the present disclosure will typically comprise an Fc region between the first antigen-binding domain and the second antigen-binding domain.

The Fc region of the bispecific antibodies of the present disclosure may be human Fc region. The Fc region of the bispecific antibodies of the present disclosure may be of any isotype, including, but not limited to, IgG1, IgG2, IgG3 or IgG4. In some embodiments, the Fc region is of the IgG1 isotype.

In the context of bispecific antibodies of the present disclosure, the Fc region may comprise one or more amino acid changes (e.g., insertions, deletions or substitutions) as compared to the specified chimeric version of the Fc region, without changing the desired functionality. For example, the disclosure includes bispecific antigen-binding molecules comprising one or more modifications in the Fc region that results in a modified Fc region having a modified binding interaction (e.g., enhanced or diminished) between Fc and FcRn. Non-limiting examples of such Fc modifications include, e.g., a mutation of serine (“S”) to proline (“P”) at position 228 of the amino acid sequence of human IgG4 Fc region.

In certain embodiments, the Fc modification comprise a LALA mutation, i.e. mutations of L234A and L235A, according to EU numbering as in Kabat et al.. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

Effect of the Bispecific Antibodies

The antibodies of the present disclosure can bind to human PD-L1 and LAG-3 protein with high affinity; have no cross-reactive binding to human PD-2 or CD4; block the binding between PD-1 and PD-L1, as well as the binding between LAG-3 and MHC-II or FGL-1; induce a higher level of cytokine (e.g. IL-2) production; and provide significantly better anti-tumor efficacy than anti-PD-L1 or anti-LAG-3 monospecific antibody alone or in combination.

Nucleic Acid Molecules Encoding Antibodies of the Disclosure

In some aspects, the disclosure is directed to an isolated nucleic acid molecule, comprising a nucleic acid sequence encoding the first heavy chain variable region and/or the second heavy chain variable region of the bispecific antibody as disclosed herein.

Specifically, the isolated nucleic acid molecule encoding the first heavy chain variable region of the antibody may comprise a nucleic acid sequence selected from the group consisting of:

(A) a nucleic acid sequence that encodes a heavy chain variable region as set forth in SEQ ID NO: 7;
(B) a nucleic acid sequence as set forth in SEQ ID NO: 9; or
(C) a nucleic acid sequence that hybridized under high stringency conditions to the complementary strand of the nucleic acid sequence of (A) or (B).

The isolated nucleic acid molecule encoding the second heavy chain variable region of the antibody may comprise a nucleic acid sequence selected from the group consisting of:

(A) a nucleic acid sequence that encodes a heavy chain variable region as set forth in SEQ ID NO: 8;
(B) a nucleic acid sequence as set forth in SEQ ID NO: 10; or
(C) a nucleic acid sequence that hybridized under high stringency conditions to the complementary strand of the nucleic acid sequence of (A) or (B).

In some aspects, the disclosure is directed to a vector comprising the nucleic acid sequence as disclosed herein. In a further embodiment, the expression vector further comprises a nucleotide sequence encoding the constant region of a bispecific antibody, e.g. a humanized bispecific antibody.

A vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In some embodiments, a nucleic acid encoding PD-L1 or LAG-3 binding domain is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaP04-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In some embodiments, the vector is suitable for expression of the anti-PD-L1/LAG-3 bispecific antibody in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison Wis.) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH promoters (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine synthetase as a selectable marker, such as the vectors described in Bebbington (1992) Biotechnology (NY) 10: 169-175.

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In an even further aspect, the disclosure relates to a host cell comprising the vector specified herein above.

Thus, the present disclosure also relates to a recombinant eukaryotic or prokaryotic host cell which produces a bispecific antibody of the present disclosure, such as a transfectoma. A bispecific antibody may be expressed in a recombinant eukaryotic or prokaryotic host cell, such as a transfectoma, which produces the bispecific antibody of the disclosure as defined herein.

Examples of host cells include yeast, bacterial, plant and mammalian cells, such as CHO, CHO-S, HEK, HEK293, HEK-293F, Expi293F, PER.C6 or NSO cells or lymphocytic cells. For example, in some embodiments, the host cell may comprise a first and second nucleic acid construct stably integrated into the cellular genome, wherein the nucleic acid construct comprises nucleic acid sequences encoding the first and second antigen binding domains as described above. In another embodiment, the present disclosure provides a cell comprising a non-integrated nucleic acid, such as a plasmid, cosmid, phagemid, or linear expression element, which comprises a first and second nucleic acid construct as specified above.

In an even further aspect, the disclosure relates to a transgenic non-human animal or plant comprising nucleic acids encoding one or two sets of a human heavy chain and a human light chain, wherein the animal or plant produces a bispecific antibody of the disclosure.

In a further aspect, the disclosure relates to a hybridoma which produces an antibody for use in a bispecific antibody as defined herein.

In one aspect, the disclosure relates to an expression vector comprising

(i) a nucleic acid sequence encoding the first antigen-binding domain according to any one of the embodiments disclosed herein;

(ii) a nucleic acid sequence encoding the second antigen-binding domain according to any one of the embodiments disclosed herein;

(iii) a nucleic acid sequence encoding a Fc region;

(iv) a nucleic acid sequence encoding a linker; or

(v) the combinations of two or more of the above.

In one aspect, the disclosure relates to a nucleic acid construct encoding one or more amino acid sequences set out in the sequence listing.

In one aspect, the disclosure relates to a method for producing a bispecific antibody according to any one of the embodiments as disclosed herein, comprising the steps of culturing a host cell comprising an expression vector or more than one expression vectors expressing the bispecific antibody as disclosed herein and purifying said antibody from the culture media. In one aspect, the disclosure relates to a host cell comprising an expression vector as defined above. In one embodiment, the host cell is a recombinant eukaryotic, recombinant prokaryotic, or recombinant microbial host cell.

Pharmaceutical Compositions

In some aspects, the disclosure is directed to a pharmaceutical composition comprising at least one antibody or antigen-binding portion thereof as disclosed herein and a pharmaceutically acceptable carrier.

Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-PD-L1/anti-LAG-3 bispecific antibody enhances the immune response. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, or other various combinations of components that are known in the art.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed herein, in a solvent containing the bispecific antibody or antigen-binding fragment thereof, one or more anti-oxidants such as methionine are included, reducing antibody or antigen binding fragment thereof that may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetra acetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers and include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight per dose.

In any event, the antibody or the antigen binding portion thereof of the disclosure is preferably administered as needed to a subject in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the instant disclosure will comprise multiple doses of the selected drug product over a period of weeks or months. More specifically, the antibody or the antigen-binding portion thereof of the instant disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments ADC concentrations will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml

### Applications of the Disclosure

In some aspects, the present disclosure provides a method of treating a disease or condition in a subject, which comprises administering to the subject (for example, a human) in need of treatment a therapeutically effective amount of the antibody or antigen-binding portion thereof as disclosed herein. For example, the disease or condition may be a cancer, an autoimmune disease or an infectious disease.

A variety of cancers where PD-L1 and/or LAG-3 is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, chondromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In some specific embodiments, the cancer is colon cancer.

In some embodiments, examples of cancer include but not limited to B-cell cancers, including B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliierative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some other embodiments, the disease or condition is an autoimmune disease. Examples of autoimmune diseases that may be treated with the antibody or antigen-binding portion thereof as disclosed herein include autoimmune encephalomyelitis, lupus erythematosus, and rheumatoid arthritis, among others. The antibody or the antigen-binding portion thereof may also be used to treat or prevent infectious disease, inflammatory disease (such as allergic asthma) and chronic graft-versus-host disease.

Combined Use with Chemotherapies

The antibody or the antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed bispecific antibodies prior to administration. More specifically, in certain embodiments selected anti-cancer agents will be linked to the unpaired cysteines of the engineered antibodies to provide engineered conjugates. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin,

Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the antibody or the antigen-binding portion thereof of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the antibody or the antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and may be used in connection with a targeted anti-cancer agent, antibody, conjugate or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In some embodiments, the composition contained in the unit dosage may comprise saline, sucrose, or the like; a buffer, such as phosphate, or the like; and/or be formulated within a stable and effective pH range. Alternatively, in certain embodiments, the antibody or composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed antibody or composition is used for treating the disease or condition of choice.

The present disclosure also provides kits for producing single-dose or multi-dose administration units of antibodies and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed bispecific antibodies or conjugates, compositions thereof. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the antibodies and, optionally, one or more anti-cancer agents in the same or different containers. The kits may also contain other pharmaceutically acceptable formulations, either for diagnosis or combined therapy. For example, in addition to the antibody or the antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

Appended to the instant application is a sequence listing comprising a number of nucleic acid and amino acid sequences. The following Tables A, B, and C provide a summary of the included sequences.

The illustrative antibody as disclosed herein, which is an anti-PD-L1/anti-LAG-3 bispecific antibody, is designated as “W3669-U15T4.G1-1.uIgG1LALA” or “W3669 antibody”.

TABLE A
CDRs of W3669-U15T4.G1-1.uIgG1LALA

| | CDR1 | CDR2 | CDR3 |
|---|---|---|---|
| Anti-PD-L1 | SEQ ID NO: 1 | SEQ ID NO: 2 | SEQ ID NO: 3 |
| VHH | GHFSNLAVN | GILWSGGSTFYA | GTN |
| | | DSVKG | |
| | CDR1 | CDR2 | CDR3 |
| Anti-LAG-3 | SEQ ID NO: 4 | SEQ ID NO: 5 | SEQ ID NO: 6 |
| VHH | GLTLSQYTMG | AIHWTSSVTDYA | THYYTHRGPF |
| | | DSVYG | DY |

TABLE B
Sequences of VHHs of W3669-U15T4.G1-1.uIgG1LALA

| | 1st VHH (anti-PD-L1) | 2nd VHH (anti-LAG-3) |
|---|---|---|
| Amino | SEQ ID NO: 7 | SEQ ID NO: 8 |
| acid | EVQLVESGGGLVQPGGSLRLSC | QVQLVESGGGVVQPGGSLRLS |
| sequence | AASGHFSNLAVNWFRQAPGKE | CAASGLTLSQYTMGWFRQAP |
| | RELVAGILWSGGSTFYADSVKG | GKERELVAAIHWTSSVTDYAD |
| | RFTISRGNAENMLYLQMNSLRA | SVYGRFTISRDDSKNTGYLQM |
| | EDTAVYYCNTGTNWGQGTLVT | NSLRAEDTAVYYCAATHYYT |
| | VSS | HRGPFDYWGQGTLVTVSS |
| Nucleotide | SEQ ID NO: 9 | SEQ ID NO: 10 |
| sequence | GAGGTGCAGCTGGTGGAGTCC | CAAGTTCAGCTGGTGGAAAG |
| | GGAGGCGGACTGGTGCAGCCT | CGGCGGTGGTGTTGTTCAGC |
| | GGAGGAAGCCTGAGACTGAGC | CGGGTGGCAGTCTGCGTCTG |
| | TGCGCCGCTAGCGGCCACTTC | AGCTGCGCAGCCAGTGGTCT |
| | AGCAACCTGGCCGTGAACTGG | GACTTTAAGCCAGTATACCA |
| | TTCAGGCAGGCCCCTGGCAAG | TGGGTTGGTTTCGCCAAGCT |
| | GAGAGGGAGCTGGTGGCTGGC | CCGGGTAAAGAACGCGAACT |
| | ATCCTGTGGAGCGGCGGAAGC | GGTGGCCGCCATTCATTGGA |
| | ACCTTCTACGCCGACAGCGTG | CCAGCAGCGTGACCGATTAT |
| | AAGGGCAGGTTCACCATCAGC | GCCGATAGCGTGTACGGCCG |
| | AGGGGCAACGCCGAGAACATG | CTTTACCATTAGCCGCGATG |
| | CTGTACCTGCAGATGAACTCC | ATAGCAAAAATACTGGTTAT |
| | CTGAGGGCCGAGGACACCGCC | CTGCAGATGAATTCTTTACG |
| | GTGTACTACTGCAACACCGGC | CGCCGAAGATACCGCCGTGT |
| | ACCAACTGGGGCCAGGGCACA | ATTACTGCGCCGCCACCCAC |
| | CTCGTGACCGTGAGCAGC | TACTATACCCATCGCGGCCC |
| | | CTTTGATTACTGGGGTCAAG |
| | | GTACTTTAGTGACCGTGAGC |
| | | AGC |

TABLE C
Other sequences of W3669-U15T4.G1-1.uIgG1LALA

| Constant region | Linker | Full length sequence |
|---|---|---|
| SEQ ID NO: 11 | SEQ ID NO: 12 | SEQ ID NO: 13 |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Benchmark Antibodies and Cell Lines

### 1.1 Preparation of Materials

Information on the commercially available materials used in the examples are provided in Table 1.

TABLE 1

| | | Catalog No. |
|---|---|---|
| Materials | Vendor | (Cat.) |
| W315-hPro1.ECD.His | Sino Biologics | Cat. 10084-H08H |
| W315-mPro1.ECD.His | Sino Biologics | Cat. 50010-M08H |
| W315-cynoPro1.ECD.His | Sino Biologics | Cat. 90251-C08H |
| Expi293 ™ Expression System Kit | Thermo Fisher | A14635 |
| | scientific | |
| Expi293F | ThermoFisher | A14527 |
| ExpiFectamine293 Transfection Kit | Invitrogen | A14524 |
| Expi293 Expression Medium | Invitrogen | A1435101 |
| Protein A column | GE Healthcare | 17543802 |
| SEC column | GE Healthcare | 28990944 |
| CellTracker ™ FarRed | Invitrogen | C34572 |
| PE-labeled goat anti-human IgG | Jackson | 109-115-098 |
| HRP-labeled goat anti-human IgG | Bethyl | A80-148P |
| antibody | | |
| Jurkat cell line | ATCC | TIB-152 |
| Raji cell line | ATCC | CCL-86 |

### 1.2 Generation of Soluble Antigens

Nucleic acid encoding human PD-L1 ECD (NP_054862.1) was synthesized by GENEWIZ (Suzhou, China), and then subcloned into modified pcDNA3.3 expression vectors with mouse Fc tag at the C-terminal. The plasmid was transfected into Expi293F cells (ThermoFisher). The cells were cultured in Expi293™ Expression Medium (ThermoFisher) at 37° C., 5% CO₂. After 5 days of culture, supernatant harvested from the culture of transiently transfected cells was used for protein purification. The fusion protein was purified by nickel, protein A and/or SEC column.

W315-hProl.ECD.His, W315-mProl.ECD.His and W315-cynoProl.ECD.His, corresponding to human, mouse and cynomolgus PD-L1 ECD protein with a his tag, were purchased from Sino Biologics.

Nucleic acid encoding human LAG-3 ECD (UniProt-P18627) was synthesized by Sangon Biotech. LAG-3 gene fragments were amplified from the synthesized nucleic acid and inserted into modified pcDNA3.3 expression vectors. Fusion protein containing human LAG-3 ECD with human Fc tag was obtained by transfection of human LAG-3 gene into Expi293F cells (ThermoFisher). The cells were cultured in Expi293™ Expression Medium (ThermoFisher) at 37° C., 5% CO₂. After 5 days of culture, supernatants harvested from the culture of transiently transfected cells were used for protein purification. The fusion protein was purified by protein A and/or SEC columns. W339-hProl.ECD, an untagged LAG-3 ECD protein, was generated by cleavage of ECD-hFc fusion protein with a cut site using Factor Xa protease (New England Biolabs).

Nucleic acid encoding mouse LAG-3 ECD (UniProt-Q61790) was synthesized by Sangon Biotech. LAG-3 gene fragments were amplified from the synthesized nucleic acid and inserted into modified pcDNA3.3 expression vectors with a His tag. W339-mProl.ECD.His was obtained by transfection of the plasmid into Expi293F cells (ThermoFisher). The cells were cultured in Expi293™ Expression Medium (ThermoFisher) at 37° C., 5% CO₂. After 5 days of culture, supernatants harvested from the culture of transiently transfected cells were used for protein purification. The protein was purified by nickel and/or SEC columns.

### 1.3 Generate BMK Antibodies

W366-BMK1 and W366-BMK2: DNA sequences encoding FS18-7-9/84G09LALA and FS18-7-108-29/S1 as disclosed in patent WO2017220569A1 [8], were synthesized by GENEWIZ (Suzhou, China), and then subcloned into a mammalian cell expression vector. FS18-7-9/84G09LALA and FS18-7-108-29/S1 are bispecific antibodies against PD-L1 and LAG-3, and are referred to as W366-BMK1 and W366-BMK2 below, respectively.

WBP315-BMK8: DNA sequences of anti-human PD-L1 benchmark antibodies (Atezolizumab) were synthesized based on the information disclosed in patent US20130045202A1, and then subcloned into pcDNA3.3 plasmid. This benchmark antibody is referred to as WBP315-BMK8 below.

WBP339-BMK1: DNA sequences of anti-human LAG-3 benchmark antibodies were synthesized based on the information (referred to as “25F7”) disclosed in patent US20110150892 A1, and then subcloned into pcDNA3.3 plasmid. This benchmark antibody is referred to as WBP339-BMK1 below.

The plasmids were transfected into Expi293 cells. Cells were cultured for 5 days and supernatant was collected for protein purification using Protein A column (GE Healthcare, 175438).

The obtained antibodies were analyzed by SDS-PAGE and SEC, and then stored at −80° C.

### 1.4 Cell Pool/Line Generation

Human PD-L1-expressing cell line (W315-CHO-K1.hProl.C11), mouse PD-L1-expressing cell line (W315-293F. mProl.C1) and cynomolgus monkey PD-L1-expressing cell line (W315-293F.cynoProl.2A2) were generated. Briefly, CHO-K1 or 293F cells were transfected with pcDNA3.3 expression vector containing full-length of human, mouse and cynomolgus PD-L1 using Lipofectamine 2000 transfection kit according to manufacturer's protocol, respectively. At 48-72 hours post transfection, the transfected cells were cultured in medium containing blasticidin for selection and tested for PD-L1 expression. Human PD-L1-expressing cell lines, cynomolgus monkey PD-L1-expressing cell lines, and mouse PD-L1 expressing cell lines were obtained by limited dilution.

Human LAG-3-expressing cell line (W339-FlpIn293.hProl.A3), mouse LAG-3-expressing cell line (W339-FlpCHO.mProl.A4) and cynomolgus LAG-3-expressing cell line (W339-293F.cProl.A4) were generated. Briefly, Flp-In-293, Flp-In-CHO or 293F cells were transfected with pcDNA3.3 expression vector containing full-length of human, mouse and cynomolgus LAG-3 using Lipofectamine 2000 transfection kit according to manufacturer's protocol, respectively. At 48-72 hours post transfection, the transfected cells were cultured in medium containing blasticidin for selection and tested for LAG-3 expression. Human LAG-3-expressing cell lines, cynomolgus monkey LAG-3-expressing cell lines, and mouse LAG-3 expressing cell lines were obtained by limited dilution.

### Example 2

### Generation of Bispecific Antibodies

Anti-PD-L1 domain antibody (VHH) and anti-LAG-3 VHH were obtained respectively by immunizing llama followed by panning and screening of the phage-displayed VHH libraries. The selected VHHs for constructing a bispecific antibody were humanized and characterized. The resulting sequences are set forth in Tables A and B.

DNA sequences encoding the humanized anti-PD-L1 VHH and anti-LAG-3 VHH were codon optimized by GENEWIZ (Suzhou, China). Then anti-PD-L1 VHH was subcloned at N-terminal of hinge region and anti-LAG-3 VHH was subcloned at C-terminal of human IgG1 Fc region with LALA mutation (L234A L235A) in a mammalian expression vector.

The plasmid of the bispecific antibody was transfected into Expi293 cells. The cells were cultured for five days and the culture supernatant was collected for protein purification using Protein A column (GE Healthcare, 175438). The obtained antibody, named W3669-U15T4.G1-1.uIgG1LALA, was analyzed by SDS-PAGE and HPLC-SEC, and then stored at −80° C.

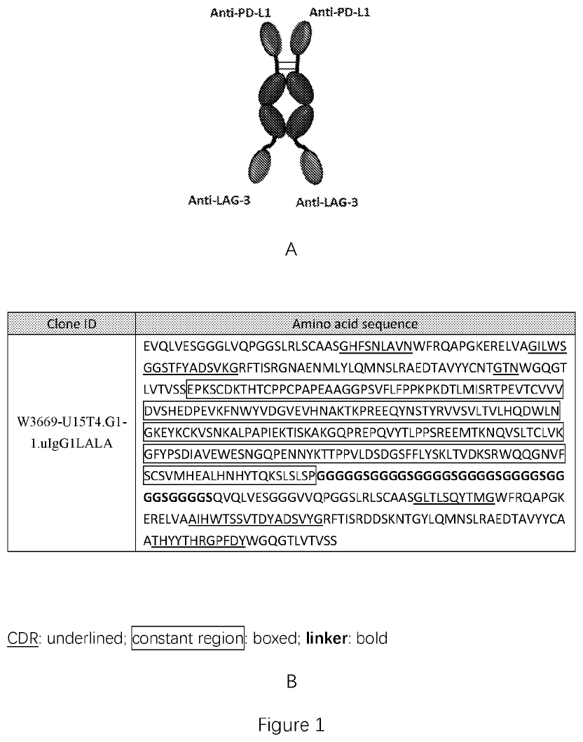

W3669-U15T4.G1-1.uIgG1LALA was also referred to as “W3669 antibody” throughout the disclosure. The schematic graph of its structure and its sequence information are shown in FIGS. 1A and 1B, respectively.

### Example 3

### In Vitro Characterization of the W3669 Bispecific Antibody

### 3.1 Purity by SEC-HPLC

The bispecific antibody W3669-U15T4.G1-1.uIgG1LALA was purified using Protein A chromatography, followed by analysis using SDS-PAGE and SEC-HPLC. The purity of the antibody was tested by SEC-HPLC using Agilent 1260 Infinity HPLC. 50 μL of antibody solution was injected on a TSKgel SuperSW3000 column using 50 mM sodium phosphate, 0.15 M NaCl, pH 7.0 buffer. The running time was 20 min. Peak retention times on the column were monitored at 280 nm. The data was analyzed using ChemStation software (V2.99.2.0).

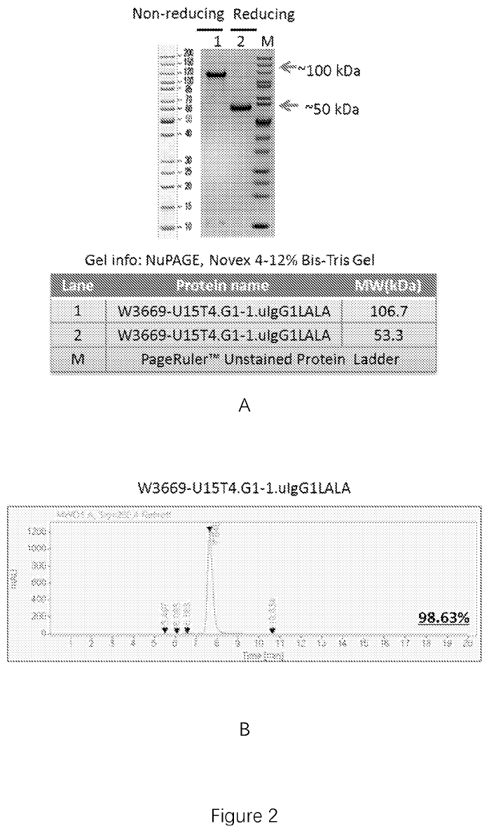

As shown in the SDS-PAGE gel (FIG. 2A), the W3669 antibody showed a band at ˜100 kDa under non-reducing condition and ˜50 kDa under reducing condition, respectively, which matches with the deduced molecular weight of the antibody. In the SEC-HPLC profile (FIG. 2B), the antibody showed symmetric single peak with calculated purity of 98.63%. Both data sets indicate that the antibody preparation had high purity.

### 3.2 Target Binding Determined by ELISA/FACS

(I) Human PD-L1 Binding

The binding of the bispecific antibodies to cell surface human PD-L1 was determined by FACS. Briefly, human PD-L1-expressing cells W315-CHO-K1.hprol.C11 were incubated with various concentrations of PD-L1×LAG-3 bispecific antibodies. PE-labeled goat anti-human IgG antibody was used to detect the binding of PD-L1×LAG-3 bispecific antibodies onto the cells. MFI of the cells was measured by flow cytometry and analyzed by FlowJo (version 7.6.1). The EC50 values of cell binding were determined using GraphPad Prism 5 software (GraphPad Software, La Jolla, Calif.).

The binding of the bispecific antibodies to Human PD-L1 protein was determined by ELISA. Briefly, plates were coated with human PD-L1 (W315-hProl.ECD.mFc) at 1 μg/mL overnight at 4° C. After blocking and washing, various concentrations of PD-L1×LAG-3 bispecific antibodies were added to the plates and incubated at room temperature for 1 hour. The plates were then washed and subsequently incubated with HRP-labeled goat anti-human IgG antibody for 1 hour. After washing, TMB substrate was added and the color reaction was stopped by 2M HCl. The absorbance at 450 nm was read using a microplate reader.

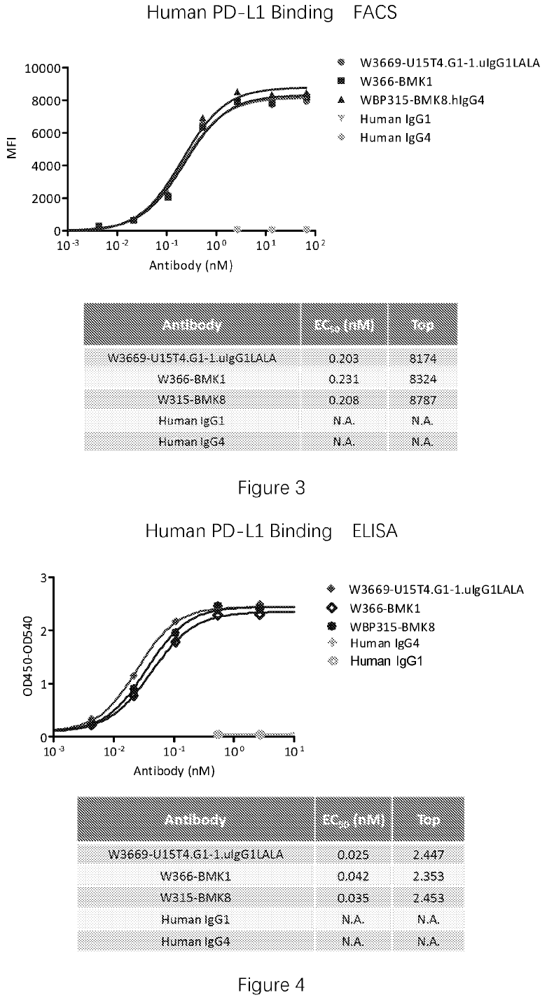

The binding results were shown in FIGS. 3 and 4. As indicated in FIG. 3, W3669 bispecific antibody bound to human PD-L1-expressed CHO-K1 cells, comparable (in EC50 and Top MFI value) with the control bispecific antibody (W366-BMK1) and anti-PD-L1 antibody (W315-BMK8, i.e. WBP315-BMK8.hIgG4 in FIG. 3).

Similarly, FIG. 4 demonstrated that the W3669 bispecific antibody bound to recombinant human PD-L1 protein, comparable (in EC50 and Top value) to the control bispecific antibody (W366-BMK1) and anti-PD-L1 antibody (W315-BMK8).

(II) Human LAG-3 Binding

The binding of the bispecific antibodies to cell surface human LAG-3 was determined by FACS. Briefly, human LAG-3 expressing cells W339-FlpIn293.hProl.A3 were incubated with various concentrations of PD-L1×LAG-3 antibodies. PE-labeled goat anti-human IgG antibody was used to detect the binding of W3669 bispecific antibody onto the cells. MFI of the cells was measured by flow cytometry and analyzed by FlowJo. The EC50 values of cell binding were determined using GraphPad Prism 5 software (GraphPad Software, La Jolla, Calif.).

The binding of the bispecific antibodies to Human LAG-3 Protein was determined by ELISA. Briefly, plates were coated with goat anti-mouse F(ab)2 at 1 μg/mL overnight at 4° C. After blocking and washing, human LAG-3 protein (W339-hProl.ECD.mFc) was added to the plates at the concentration of 1 μg/mL. Various concentrations of PD-L1×LAG-3 bispecific antibodies were added to the plates and incubated at room temperature for 1 hour after washing. The plates were then washed and subsequently incubated with HRP-labeled goat anti-human IgG antibody for 1 hour. After washing, TMB substrate was added and the color reaction was stopped by 2M HCl. The absorbance at 450 nm was read using a microplate reader.

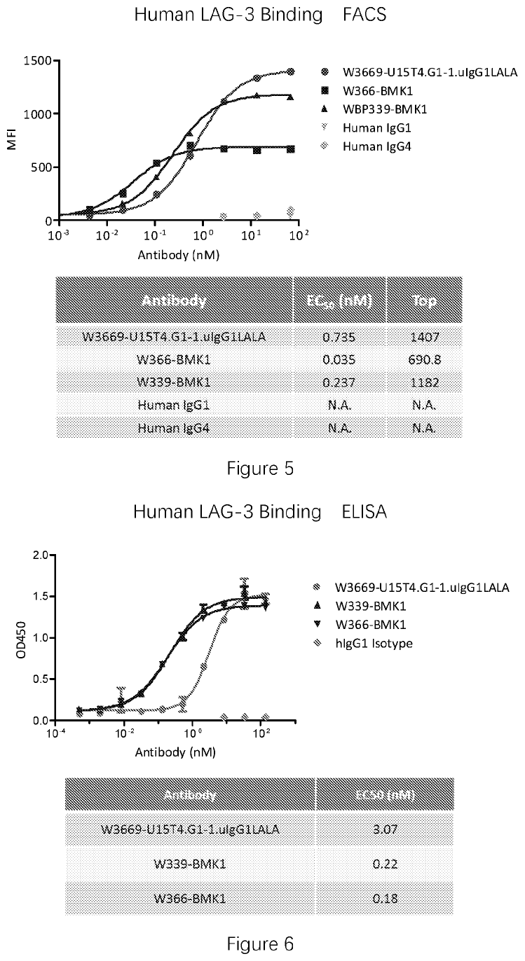

The results were shown in FIGS. 5 and 6. As shown in FIG. 5, the W3669 bispecific antibody bound to human LAG-3 transfected 293 cells, with higher Top MFI value than the control bispecific antibody (W366-BMK1) and anti-LAG-3 antibody (W339-BMK1, i.e. WBP339-BMK1 in FIG. 5).

In addition, FIG. 6 shows that the W3669 bispecific antibody bound to recombinant human LAG-3 protein. However, the binding of W3669 antibody was weaker than that of bispecific BMK (W366-BMK1) and anti-LAG-3 BMK (W339-BMK1), possibly because epitopes recognized by W3669 antibody were altered on ELISA plate.

(III) Dual Binding to Human PD-L1 and LAG-3

Dual Binding to human PD-L1 and LAG-3 proteins was determined by ELISA. Plates were coated with mouse anti-His antibody at 1 μg/ml overnight at 4° C. After blocking and washing, human LAG-3 protein (W339-hProl.ECD.His) was added to the plates at the concentration of 1 μg/mL. Various concentrations of PD-L1×LAG-3 antibodies were added to the plates and incubated at room temperature for 1 hour after washing. The plates were then washed and subsequently incubated with biotin-labeled mouse Fc tagged PD-L1 protein (W315-hProl.ECD.mFc) for 1 hour. After washing, HPR-conjugated streptavidin was added to the plate and incubated at room temperature for 0.5 hour. After washing, TMB substrate was added and the color reaction was stopped by 2M HCl. The absorbance at 450 nm was read using a microplate reader.

Dual binding to cell surface human PD-L1 and LAG-3 was determined by FACS. Human PD-L1 expressing cells W315-CHO-K1.hprol.C11 and human LAG-3 transiently transfected cells W339-293F.hProl were stained with Far Red (Invitrogen-C34572) and CFSE (Invitrogen-C34554), respectively. The two types of cells were co-incubated with the presence of various concentrations of PD-L1×LAG-3 antibodies or control antibodies for 2 hrs at 4° C. The percentage of the cells linked with antibodies (double positive) was measured by flow cytometry and analyzed by FlowJo (version 7.6.1).

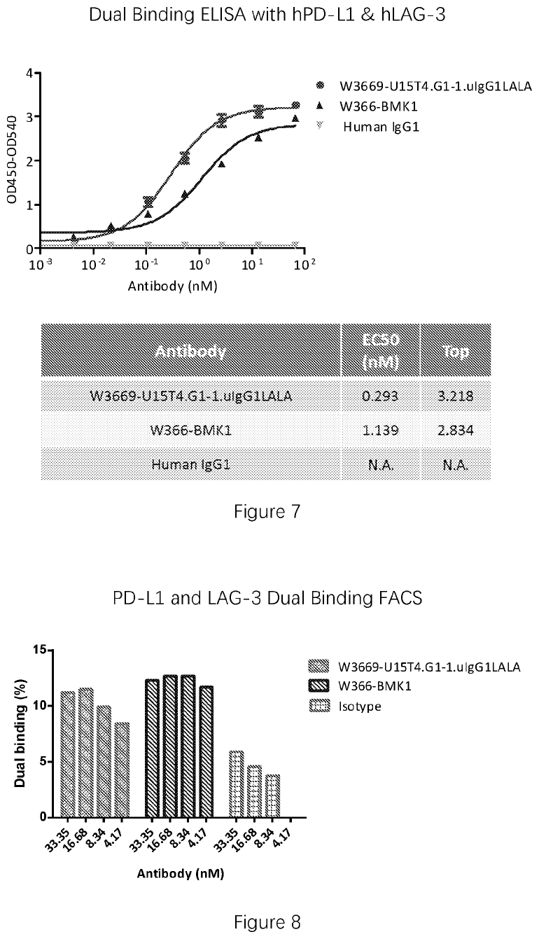

The results were shown in FIGS. 7 and 8. FIG. 7 indicated that the W3669 bispecific antibody and the bispecific W366-BMK1 bound to human PD-L1 and LAG-3 protein simultaneously and the W3669 bispecific antibody clearly showed a better performance than W366-BMK1 in EC50 and Top (i.e. max of the y axis OD450 value). FIG. 8 indicated that they also bound to human PD-L1 expressing cells and LAG-3 expressing cells simultaneously.

### 3.3 Paralog-Binding Determined by ELISA/FACS

The cross-reactivity of the bispecific antibodies to human PD-L2 was measured by FACS, in a same procedure as described above. Briefly, human PD-L2 expressing CHO-K1 cells were incubated with various concentrations of W3669 bispecific antibody at room temperature for 1 hour. PE-labeled goat anti-human IgG antibody was used to detect the binding of W3669 bispecific antibody onto the cells. MEI of the cells was measured by flow cytometry and analyzed by FlowJo.

The cross-reactivity to human CD4 was also measured by ELISA. Plates were coated with human CD4 at 1 μg/ml overnight at 4° C. After blocking and washing, various concentrations of W3669 bispecific antibody were added to the plates and incubated at room temperature for 1 h. The plates were then washed and subsequently incubated with corresponding secondary antibody for 60 min. After washing, TMB substrate was added and the color reaction was stopped by 2M HCl. The absorbance at 450 nm was read using a microplate reader.

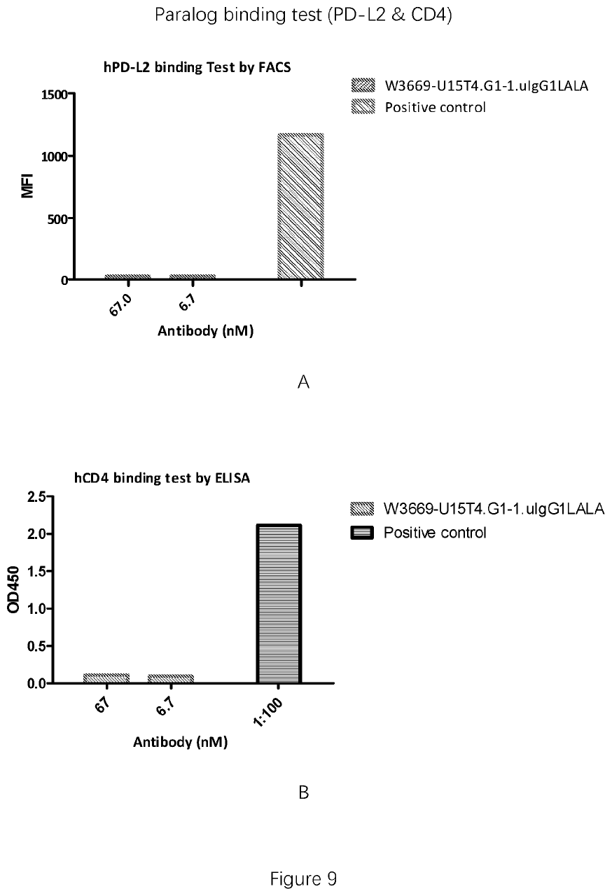

The cross-reactivity of the W3669 bispecific antibody to human PD-L2 and human CD4, measured by FACS and ELISA, were shown in FIGS. 9A and 9B, respectively. The result indicated that the W3669 bispecific antibody could not bind to human PD-L2 or human CD4, whether at low or high concentrations.

### 3.4 Cross Species Target Binding Determined by ELISA/FACS

### (I) Binding to Cyno or Mouse PD-L1

Plates were coated with cynomolgus PD-L1 (W315-cynoProl.ECD.His) or mouse PD-L1 (W315-mProl.ECD.mFc) at 1 μg/mL overnight at 4° C. After blocking and washing, various concentrations of W3669 bispecific antibody were added to the plates and incubated at room temperature for 1 hour. The plates were then washed and subsequently incubated with HRP-labeled goat anti-human IgG antibody for 1 hour. After washing, TMB substrate was added and the color reaction was stopped by 2M HCl. The absorbance at 450 nm was read using a microplate reader.

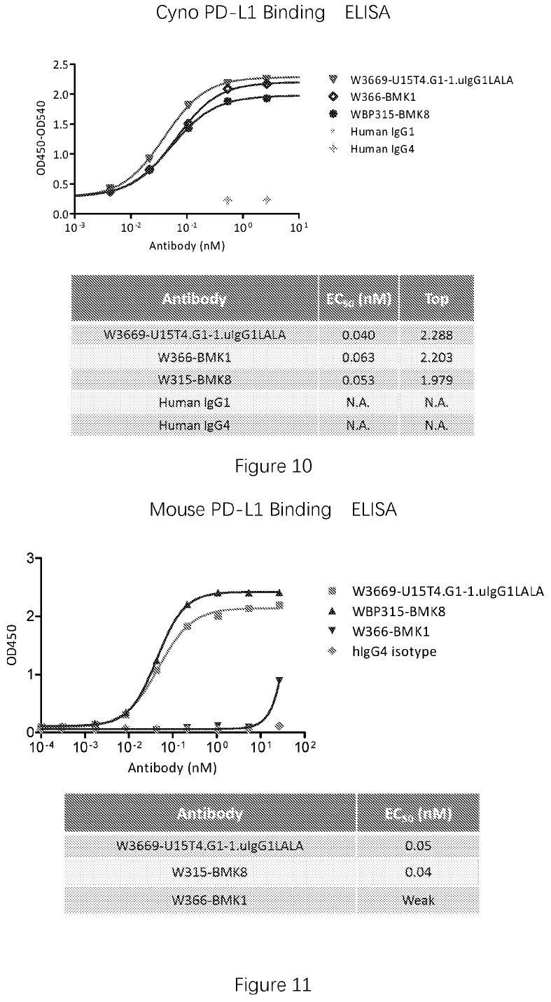

The results were shown in FIGS. 10 and 11, respectively. FIG. 10 indicated that the W3669 bispecific antibody bound to recombinant cyno PD-L1 protein, comparable with the control bispecific antibody (W366-BMK1) and anti-PD-L1 antibody (W315-BMK8). FIG. 11 indicated that the W3669 bispecific antibody and anti-PD-L1 antibody (W315-BMK8) bound to recombinant mouse PD-L1 protein, whereas the bispecific BMK (W366-BMK1) only shows weak binding at high concentration.

### (II) Binding to Cyno or Mouse LAG-3

Plates were coated with mouse anti-His at 1 μg/mL overnight at 4° C. After blocking and washing, cynomolgus LAG-3 protein (Sino-90841-C08H) or mouse LAG-3 protein (W339-mProl.ECD.His) was added to the plates at the concentration of 1 μg/mL. Various concentrations of PD-L1×LAG-3 antibodies were added to the plates and incubated at room temperature for 1 hour after washing. The plates were then washed and subsequently incubated with HRP-labeled goat anti-human IgG antibody for 1 hour. After washing, TMB substrate was added and the color reaction was stopped by 2M HCl. The absorbance at 450 nm was read using a microplate reader.

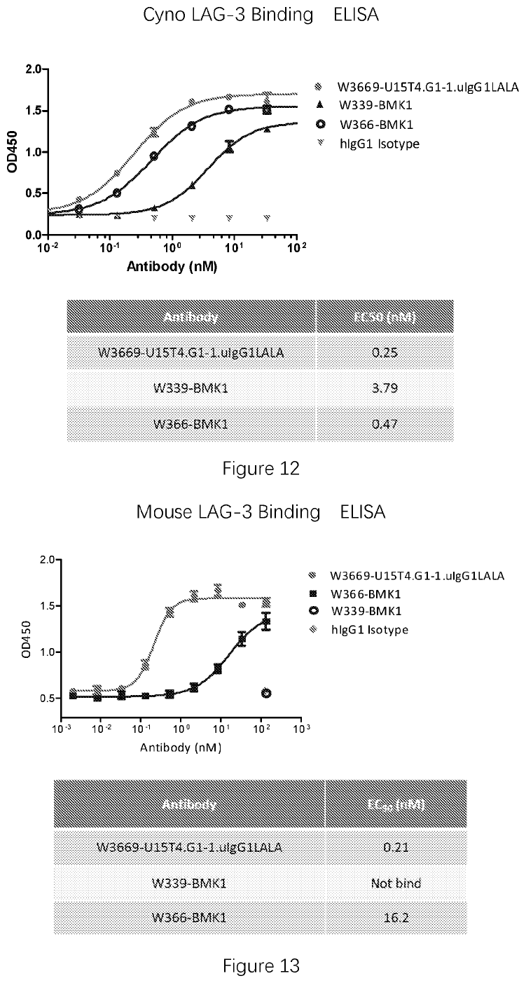

The results were shown in FIGS. 12 and 13. As shown in FIG. 12, the W3669 bispecific antibody bound to recombinant cyno LAG-3 protein, slightly better than the control bispecific antibody (W366-BMK1) and significantly better than the anti-LAG-3 antibody (W339-BMK1). As shown in FIG. 13, the W3669 bispecific antibody bound to recombinant mouse LAG-3 protein, whereas the control bispecific antibody (W366-BMK1) showed weak binding. The control anti-LAG-3 antibody (W339-BMK1) could not bind to mouse LAG-3 protein.

### 3.5 Affinity Measured by Surface Plasmon Resonance (SPR)

Antibody binding affinity to human, mouse and cyno PD-L1 as well as human and mouse LAG-3 proteins was detected by SPR assay using Biacore 8K. Each antibody was captured on an anti-human IgG Fc antibody immobilized CM5 sensor chip (GE). Antigens at different concentrations were injected over the sensor chip at a flow rate of 30 uL/min. The chip was regenerated by 10 mM pH1.5 Glycine after each binding cycle. The sensorgrams of blank surface and buffer channel were subtracted from the test sensorgrams. The experimental data was fitted by 1:1 model using Langmiur analysis. Molecular weight of 50, 50, 40, 40 and 40 kDa was used to calculate the molar concentration of analyte human, mouse and cyno PD-L1 and human and mouse LAG-3 proteins. The result was shown in Table 2 below:

TABLE 2
Affinity measured by SPR

| Antigen | Antibody | ka (1/Ms) | kd (1/s) | KD (M) |
|---|---|---|---|---|
| Human | W3669-U15T4.G1- | 5.22E+04 | 2.30E−05 | 4.41E−10 |
| LAG-3 | 1.uIgG1LALA | | | |
| | W366-BMK1 | 1.43E+06 | 1.02E−02 | 7.11E−09 |
| | W339-BMK1 | 5.08E+05 | 3.15E−04 | 6.20E−10 |
| Mouse | W3669-U15T4.G1- | 1.74E+06 | <1E−05 | <5.75E−12 |
| LAG-3 | 1.uIgG1LALA | | | |
| | W366-BMK1 | No/weak binding | | |
| Human | W3669-U15T4.G1- | 8.12E+05 | 2.84E−04 | 3.50E−10 |
| PD-L1 | 1.uIgG1LALA | | | |
| | W366-BMK1 | 8.82E+05 | 2.63E−04 | 2.98E−10 |
| | W315-BMK8 | 8.33E+05 | 6.61E−05 | 7.94E−11 |
| Mouse | W3669-U15T4.G1- | 4.89E+05 | 1.01E−02 | 2.07E−08 |
| PD-L1 | 1.uIgG1LALA | | | |
| | W366-BMK1 | No/weak binding | | |
| | W315-BMK8 | 6.67E+05 | 1.54E−03 | 2.31E−09 |
| Cyno | W3669-U15T4.G1- | 8.84E+05 | 4.58E−04 | 5.18E−10 |
| PD-L1 | 1.uIgG1LALA | | | |
| | W366-BMK1 | 8.05E+05 | 4.46E−04 | 5.54E−10 |
| | W315-BMK8 | 7.43E+05 | 4.67E−03 | 6.29E−09 |

As demonstrated in the above table, the W3669 bispecific antibody can bind to human, mouse & cyno PD-L1 and human & mouse LAG-3 with high affinities.

### 3.6 Ligand Competition Assays

Blocking of PD-1 Protein Binding to PD-L1 Expressing Cells:

The antibodies were serially diluted in 1% BSA-PBS and mixed with mFc-tagged PD-1 protein at 4° C. The mixture was transferred into the 96-well plates seeded with PD-L1 positive W315-CHO-K1.hprol.C11 cells. Goat anti-mouse IgG Fc-PE antibody was used to detect the binding of PD-1 protein to PD-L1 expressing cells. The MFI was evaluated by flow cytometry and analyzed by the software FlowJo.

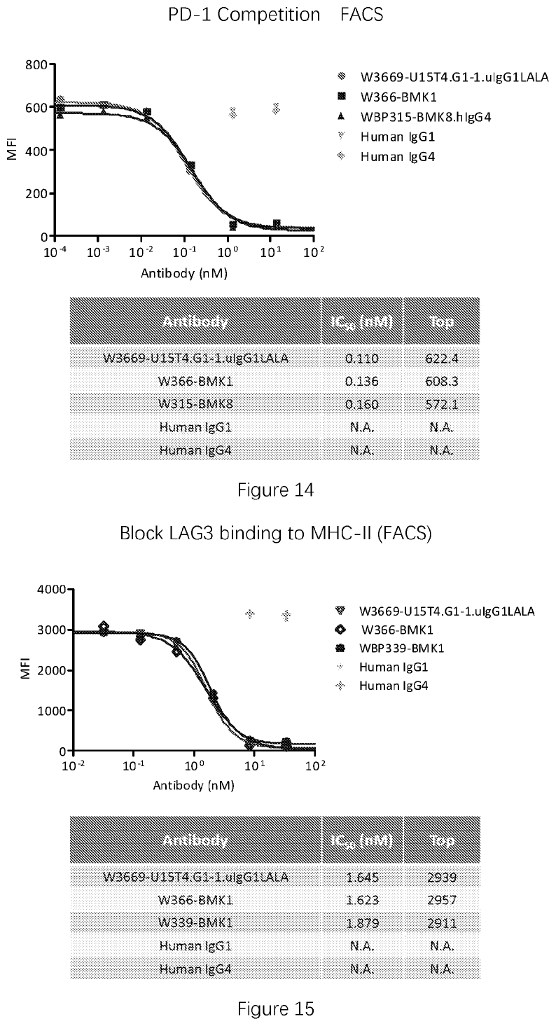

As shown in FIG. 14, the W3669 bispecific antibody, control bispecific BMK (W366-BMK1), and anti-PD-L1 antibody (W315-BMK8) blocked PD1 binding to PD-L1 expressing cells. The W3669 bispecific antibody obtained a slightly better performance in IC50 and MFI max value (i.e. Top).

Blocking of LAG-3 Protein Binding to MHC-II Expressed on Raji Cells:

Antibodies were serially diluted in 1% BSA-PBS and incubated with mFc-tagged LAG-3 protein at 4° C. for 30 min. The mixture was transferred into the 96-well plates seeded with Raji cells. Goat anti-mouse IgG Fc-PE antibody was used to detect the binding of LAG-3 protein to Raji cells. The MFI was evaluated by flow cytometry and analyzed by the software FlowJo.

As shown in FIG. 15, the W3669 bispecific antibody, the bispecific BMK (W366-BMK1), and the LAG-3 BMK (W339-BMK1) blocked LAG-3 protein binding to MHC-II expressing Raji cells.

Blocking of LAG-3 Protein Binding to FGL-1:

96-well plates were coated with human FGL-1 (SB-13484-H08B) at 0.5 μg/mL overnight at 4° C. Antibodies were serially diluted in PBS and mixed with mouse Fc-tagged LAG-3 protein. After blocking and washing, the mixture was transferred to the plates and incubated at room temperature for 1 h. The plates were then washed and subsequently incubated with HRP-labeled anti-mouse IgG antibody for 1 h. After washing, TMB substrate was added and the color reaction was stopped by 2M HCl. The absorbance at 450 nm was read using a microplate reader.

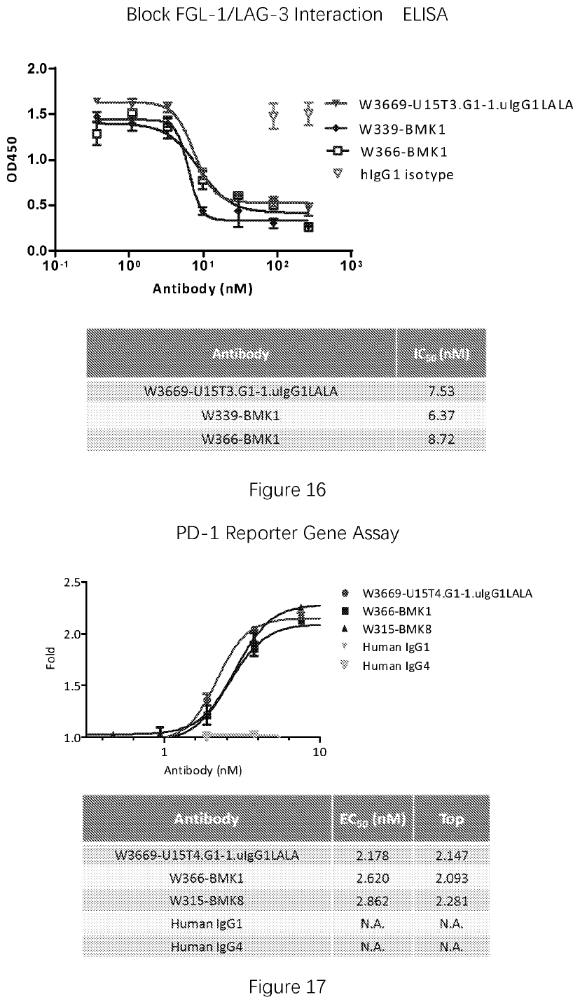

As shown in FIG. 16, the W3669 bispecific antibody, the bispecific BMK (W366-BMK1), and the LAG-3 BMK (W339-BMK1) blocked LAG-3 protein binding to FGL-1 protein. The IC50 values of PD-L1 and LAG-3 blocking were summarized in the table below.

TABLE 3

| | IC50 (nM) | IC50 (nM) | IC50 (nM) |
|---|---|---|---|
| Antibody | PD1 | MHC-II | FGL-1 |
| W3669 bispecific antibody | 0.110 | 1.645 | 7.53 |
| W366-BMK1 (bispecific) | 0.136 | 1.623 | 8.72 |
| W315-BMK8 (anti-PD-L1) | 0.160 | — | — |
| W339-BMK1 (anti-LAG-3) | — | 1.879 | 6.37 |

### 3.7 Cell-Based Functional Assays

Effects of Anti-PD-L1×LAG-3 Bispecific Antibodies in PD-1-NFAT Reporter Gene Assay:

Jurkat cells expressing human PD-1 along with stably integrated NFAT luciferase reporter gene were seeded in 96-well plates along with PD-L1 expressing artificial antigen presenting cells (APC). Different antibodies were incubated with PD-L1+ artificial antigen presenting cells and PD-1+ Jurkat cells that stably integrated NFAT luciferase reporter gene. The plates were incubated for 6 hours at 37° C., 5% CO2. After incubation, reconstituted luciferase substrate was added and the luciferase intensity was measured by a microplate spectrophotometer.

As shown in FIG. 17, the W3669 bispecific antibody, the control bispecific antibody (W366-BMK1), and the anti-PD-L1 antibody (W315-BMK8) induced the NFAT expression, indicating that these antibodies induced PD-1 signaling pathway.

Effects of Anti-PD-L1×LAG-3 Bispecific Antibodies in LAG-3-IL-2 Reporter Gene Assay:

Jurkat cells expressing human LAG-3 along with stably integrated IL-2 luciferase reporter gene were seeded in 96-well plates along with Raji cells in the presence of SEE (Staphylococcal enterotoxin E). Different antibodies were added into the system and the plates were incubated for overnight at 37° C., 5% CO2. After incubation, reconstituted luciferase substrate was added and the luciferase intensity related to IL-2 gene expression was measured by a microplate spectrophotometer.

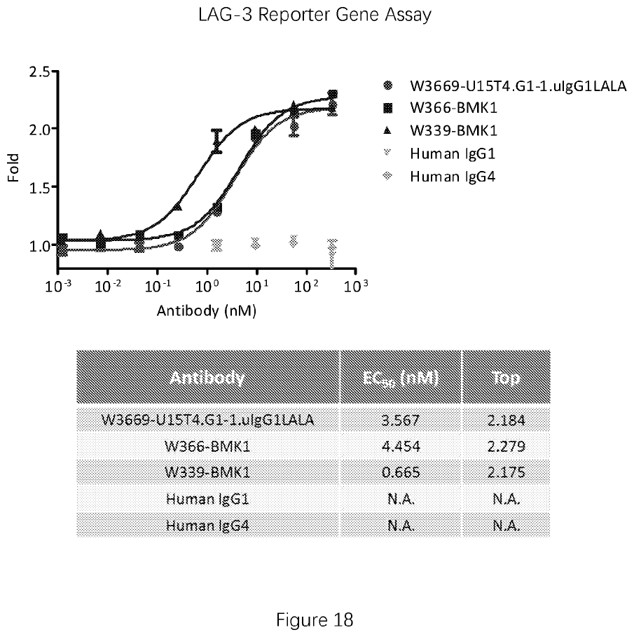

As shown in FIG. 18, W3669 bispecific antibody, the bispecific BMK (W366-BMK1), and the LAG-3 BMK (W339-BMK1) induced the IL-2 expression, indicating they induced LAG-3 signaling pathway.

Effects of Anti-PD-L1×LAG-3 Bispecific Antibodies on PD-1 and LAG-3 Expressing IL-2 Reporter Gene Assay:

Full human LAG-3 plasmid was transiently transfected into Jurkat cells expressing human PD-1 along with stably integrated NFAT luciferase reporter gene. After 48 hours, the cells were seeded in 96-well plates along with Raji cells in the presence of SEE (Staphylococcal enterotoxin E). Different antibodies were added into the system to measure their effect on IL2 expression. The plates were incubated for overnight at 37° C., 5% CO2. After incubation, reconstituted luciferase substrate was added and the luciferase intensity was measured by a microplate spectrophotometer.

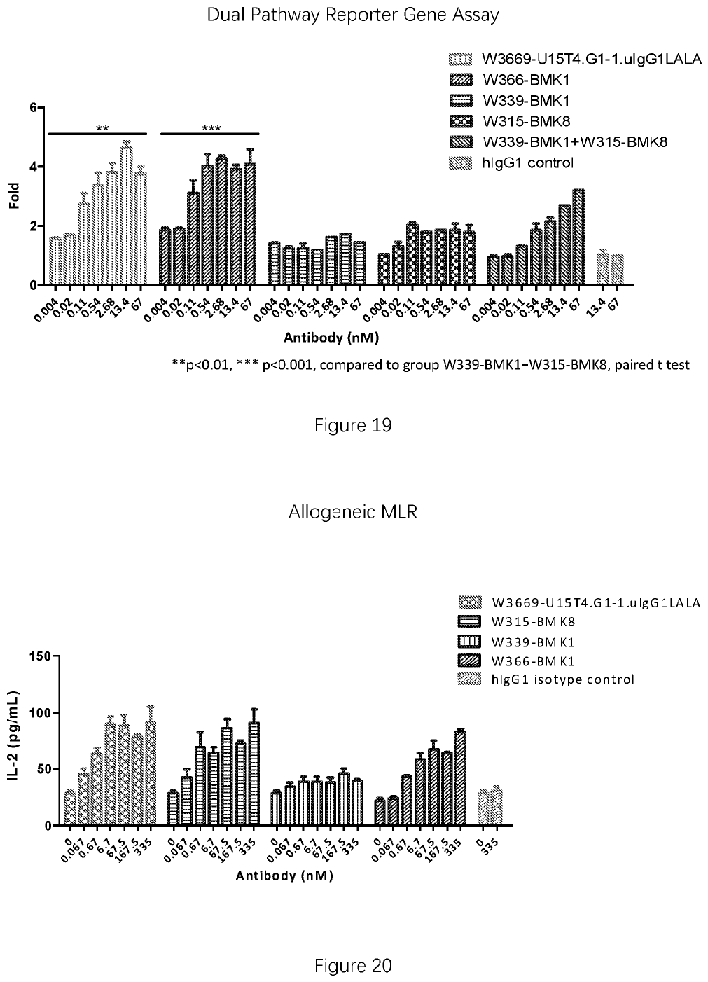

As shown in FIG. 19, the W3669 bispecific antibody and the bispecific BMK (W366-BMK1) induced significantly higher level of IL-2 expression fold change, compared with anti-LAG-3 antibody (W339-BMK1) and anti-PD-L1 antibody (W315-BMK8) and their combination.

Effects of Anti-PD-L1×LAG-3 Bispecific Antibodies on Human Allogeneic Mixed Lymphocyte Reaction:

Human peripheral blood mononuclear cells (PBMCs) were freshly isolated from healthy donors using Ficoll-Paque PLUS gradient centrifugation. Monocytes were isolated using human monocyte enrichment kit according to the manufacturer's instructions. Cells were cultured in medium containing GM-CSF and IL-4 for 5 to 7 days to generate dendritic cells (DC). Human CD4+ T cells were isolated using human CD4+ T cell enrichment kit according to the manufacturer's protocol. Purified CD4+ T cells were co-cultured with allogeneic immature DCs (iDCs) in the presence of various concentrations of W3669 bispecific antibody in 96-well plates. The plates were incubated at 37° C., 5% CO2. Supernatants were harvested for IL-2 and IFN-γ test at day 3 and day 5, respectively. Human IL-2 and IFN-γ release were measured by ELISA using matched antibody pairs. Recombinant human IL-2 and IFN-γ were used as standards, respectively. The plates were pre-coated with capture antibody specific for human IL-2 or IFN-γ, respectively. After blocking, 50 μL of standards or samples were pipetted into each well and incubated for 2 hours at ambient temperature. Following removal of the unbound substances, the biotin-conjugated detecting antibody specific for corresponding cytokine was added to the wells and incubated for one hour. HRP-streptavidin was then added to the wells for 30 minutes incubation at ambient temperature. The color was developed by dispensing 50 μL of TMB substrate, and then stopped by 50 μL of 2N HCl. The absorbance was read at 450 nM using a Microplate Spectrophotometer.

As shown in FIG. 20, the W3669 bispecific antibody, the bispecific BMK (W366-BMK1) and the PD-L1 BMK (W315-BMK8) enhanced IL-2 production in the human allogeneic mixed lymphocyte reaction.

Effects of Anti-PD-L1×LAG-3 Bispecific Antibodies on Human PBMCs Activation:

Human PBMCs and various concentrations of different antibodies were co-cultured in 96-well plates in the presence of SEB. The plates were incubated at 37° C., 5% CO2. Supernatants were harvested for IL-2 test at day 3. Human IL-2 release was measured by ELISA using matched antibody pairs. Recombinant human IL-2 was used as standards, respectively. The plates were pre-coated with capture antibody specific for human IL-2, respectively. After blocking, 50 μL of standards or samples were pipetted into each well and incubated for 2 hours at ambient temperature. Following removal of the unbound substances, the biotin-conjugated detecting antibody specific for corresponding cytokine was added to the wells and incubated for one hour. HRP-streptavidin was then added to the wells for 30 minutes incubation at ambient temperature. The color was developed by dispensing 50 μL of TMB substrate, and then stopped by 50 μL of 2N HCl. The absorbance was read at 450 nM using a Microplate Spectrophotometer.

Different antibodies were added into human PBMCs stimulated with staphylococcal enterotoxin B (SEB), then IL2 expression was measured to indicate the T cell activation.

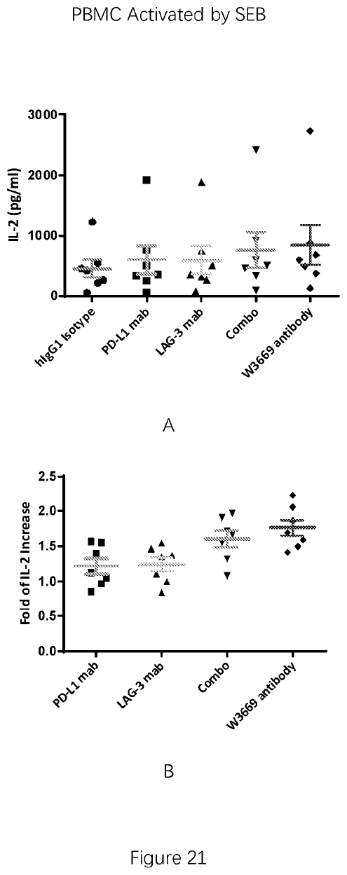

The secreted IL-2 level and fold change compared with isotype control were shown in FIGS. 21A and 21B. As shown in the figures, the W3669 bispecific antibody and PD-L1+LAG-3 mab combination (referred to as “combo” in FIG. 21) enhanced the IL-2 secretion by human PBMC stimulated with SEB, indicating the activation of T cells. The result indicated that W3669 bispecific antibody provides a better efficacy in regulating IL-2 productions.

3.8 Thermal stability measured by DSF

Tm of antibodies was investigated using QuantStudio™ 7 Flex Real-Time PCR system (Applied Biosystems). 19 μL of antibody solution was mixed with 1 μL of 62.5×SYPRO Orange solution (Invitrogen) and transferred to a 96 well plate (Biosystems). The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tm. If a protein has multiple unfolding transitions, the first two Tm were reported, named as Tm1 and Tm2. Data collection and Tm calculation were conducted automatically by the operation software.

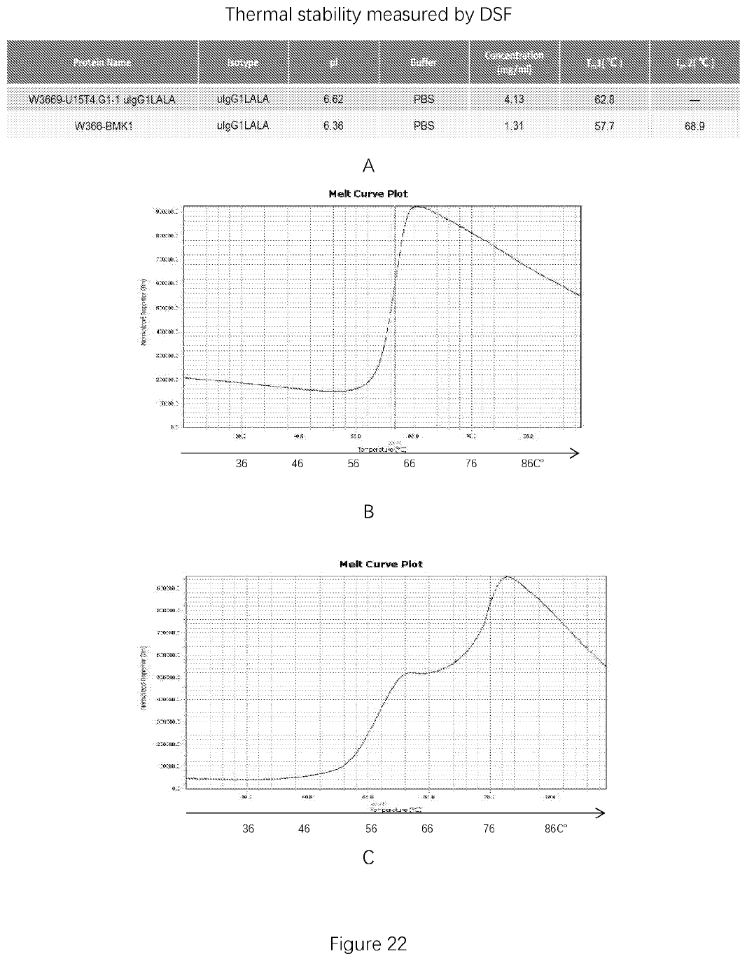

The thermal stability of the W3669 bispecific antibody and the W366-BMK1 were measured by the differential scanning fluorimetry (FIG. 22A). The Tm1 of the W3669 bispecific antibody was 62.8° C. (FIG. 22B), while the Tm1 of W366-BMK1 was 57.7° C. (FIG. 22C). The higher Tm1 of the W3669 bispecific antibody indicated better thermal stability than the bispecific W366-BMK1.

### 3.9 Serum Stability

The W3669 bispecific antibody was incubated in freshly isolated human serum (serum content>95%) at 37° C. At indicated time points, an aliquot of serum treated sample were removed from the incubator and snap frozen in liquid N2, and then stored at −80° C. until ready for test. The samples were quickly thawed immediately prior to the stability test.

For aliquots taken at different time points, the dual binding to human PD-L1 and LAG-3 proteins was tested by ELISA. Plates were coated with mouse anti-His antibody at 1 μg/ml overnight at 4° C. After blocking and washing, human LAG-3 protein (W339-hProl.ECD.His) was added to the plates at the concentration of 1 μg/mL. Various concentrations of W3669 bispecific antibody was added to the plates and incubated at room temperature for 1 hour after washing. The plates were then washed and subsequently incubated with biotin-labeled mouse Fc tagged PD-L1 protein (W315-hProl.ECD.mFc) for 1 hour. After washing, HPR-conjugated streptavidin was added to the plate and incubated at room temperature for 0.5 hour. After washing, TMB substrate was added and the color reaction was stopped by 2M HCl. The absorbance at 450 nm was read using a microplate reader.

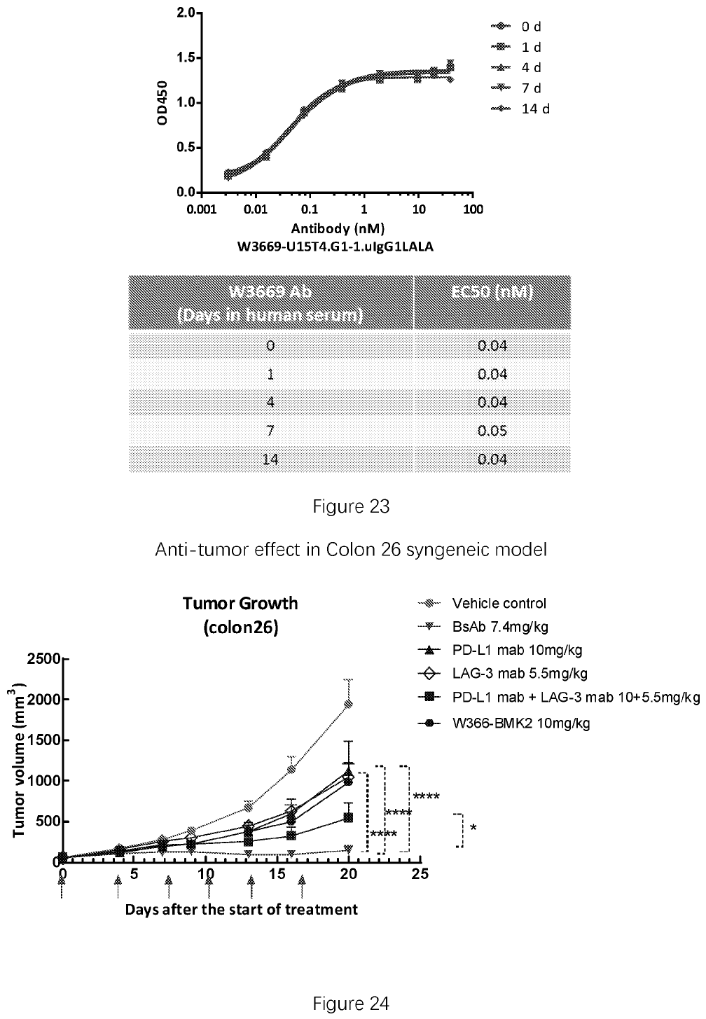

As shown in FIG. 23, the W3669 bispecific antibody exhibited normal dual binding even after 14 days incubation in human serum, which indicates the W3669 bispecific antibody was stable in human serum for at least two weeks.

### Example 4

### In Vivo Characterization of the Bispecific Antibody

### 4.1 Anti-Tumor Efficacy Study in Colon-26 Syngeneic Model

The Colon-26 tumor cells were maintained in vitro as a monolayer culture in DMEM supplemented with 10% fetal bovine serum, 100 U/ml penicillin and 100 μg/ml streptomycin at 37° C. in an atmosphere of 5% CO2 in air. The cells growing in an exponential growth phase were harvested and counted for tumor inoculation. Each mouse was inoculated subcutaneously at the right axillary (lateral) with Colon-26 tumor cell (5×10⁵) in 0.1 ml of PBS for tumor development. The animals were randomly grouped when the average tumor volume reached 60-70 mm³, then treated with the antibodies by I.P, BIW×3. Tumor sizes were measured three times weekly in two dimensions using a caliper, and the volume was expressed in mm3 using the formula: V=0.5 a×b² where a and b are the long and short diameters of the tumor, respectively.

The result was shown in FIG. 24. In the Colon 26 syngeneic model, after 6 equal molar doses of the W3669 bispecific antibody (referred to as “BsAb” in FIG. 24), the tumor volume was significantly reduced compared with anti-PD-L1 mab, anti-LAG-3 mab, anti-PD-L1 mab+anti-LAG-3 mab (referred to as “combo” in FIG. 24), or the bispecifc control (W366-BMK2) group, indicating that the W3669 bispecific antibody had superior anti-tumor effect compared to anti-PD-L1, anti-LAG-3 monotherapy, or their combined treatment. The arrows indicated the time points of administration.

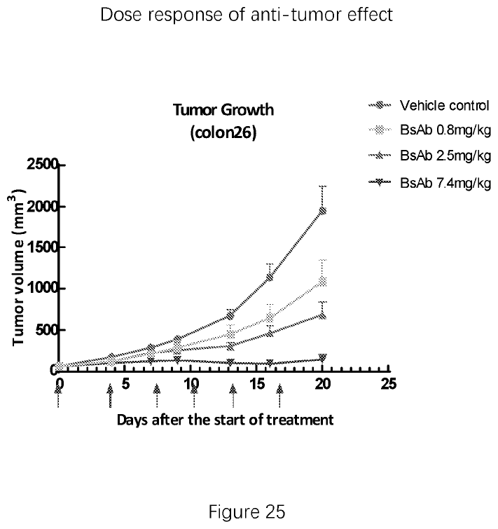

Furthermore, in the Colon 26 syngeneic model, the W3669 bispecific antibody inhibited tumor growth in a dose-response manner, as shown in FIG. 25.

### 4.2 Mouse Pharmacokinetics (PK)

Female C57BL/6 mice (Shanghai SIPPR-BK Co., Ltd) of 30-32 weeks-old were used in the study. Six animals (three animals/group) were divided into two groups: low and high dose groups. The animals in low and high dose groups were administered once with W3669-U15T4.G1-1.uIgG1LALA at 1 and 10 mg/kg, respectively. The injection was by intravenous bolus administration. The formulations were formulated in PBS. PK blood samples were collected 0.5h, 2h, 6h, 24h, Day 2, Day 4 and Day 7.

Antidrug antibody (ADA) samples were collected on 7d. Plasma samples were then prepared by centrifuging the blood samples at approximately 4° C., 5000 g for 5 minutes. All serum samples were then quickly frozen over dry ice and kept at −80° C. until ELISA analysis. Plasma concentrations of W3669-U15T4.G1-1.uIgG1LALA and ADA in plasma samples were determined by ELISA. The plasma concentration of W3669-U15T4.G1-1.uIgG1LALA in mouse was subjected to a non-compartmental pharmacokinetic analysis by using the Phoenix WinNonlin software (version 8.1, Pharsight, Mountain View, Calif.). The linear/log trapezoidal rule was applied in obtaining the PK parameters.

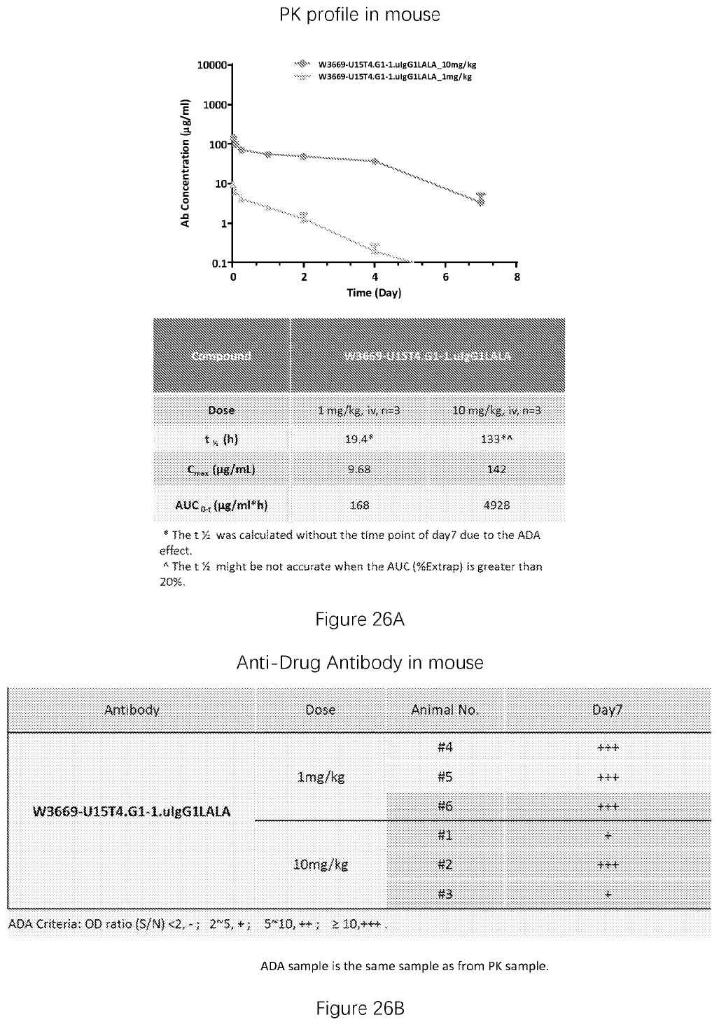

The result of the PK profile in mouse was shown in FIG. 26A. The t 1/2 of the antibody at 1 mg/kg group was 19.4 hours, and the t 1/2 of the antibody at 10 mg/kg was 133 hours, indicating that the W3669 antibody has normal PK profile in mouse at high dose (FIG. 26A).

ADA was measured using the samples taken on Day 7. ADA was observed at low dose group. As shown in FIG. 26B, all of the mice generated ADA, and 3/3 mice at low dose group had high titer, whereas only 1/3 mice at high dose group had high titer ADA.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

### REFERENCES

[1] Alsaab H O, Sau S, Alzhrani R, et al. PD-1 and PD-L1 Checkpoint Signaling Inhibition for Cancer Immunotherapy: Mechanism, Combinations, and Clinical Outcome. Frontiers in Pharmacology 2017; 8: 561.
[2] Francisco L M, Sage P T, Sharpe A H. The PD-1 pathway in tolerance and autoimmunity. Immunological Reviews 2010; 236: 219-42.
[3] Gong, Jun, Chehrazi-Raffle, Alexander et al. Development of PD-1 and PD-L1 inhibitors as a form of cancer immunotherapy: a comprehensive review of registration trials and future considerations. Journal for Immunotherapy of Cancer 2018; 6: 8.
[4] Monica V. Goldberg, Charles G. Drake. LAG-3 in Cancer Immunotherapy. Curr Top Microbiol Immunol. 2011; 344: 269-278.
[5] Lawrence P. Andrews, Ariel E. Marciscano, Charles G. Drake, et al. LAG-3 (CD223) as a cancer immunotherapy target. Immunol Rev. 2017; 276(1):80-96.
[6 Nguyen L T, Ohashi P S. Clinical blockade of PD1 and LAG-3—potential mechanisms of action. Nature Reviews Immunology. 2015 January; 15(1): 45-56.
[7] Hannah Christina Puhr, Aysegül Ilhan-Mutlu. New emerging targets in cancer immunotherapy: the role of LAG-3. ESMO Open 2019; 4:e000482.
[8] WO2017220569A1. Jamie Campbell. Binding molecules binding pd-11 and lag-3. Published 2017-12-28, assigned to F-Star Delta Limited.

1. A bispecific antibody or an antigen-binding portion thereof, comprising a first antigen binding domain and a second antigen binding domain, wherein:
the first antigen binding domain comprises: a CDRH1 comprising SEQ ID NO: 1, a CDRH2 comprising SEQ ID NO: 2, and a CDRH3 comprising SEQ ID NO: 3;
and
the second antigen binding domain comprises: a CDRH1 comprising SEQ ID NO: 4, a CDRH2 comprising SEQ ID NO: 5, and a CDRH3 comprising SEQ ID NO: 6.

2. (canceled)

3. The bispecific antibody or the antigen-binding portion thereof of claim 1, wherein:
the first antigen binding domain comprises a first VHH comprising the amino acid sequence of SEQ ID NO: 7 or a homologous sequence thereof having at least 80% sequence identity yet retaining specific binding affinity to PD-L1; and
the second antigen binding domain comprises a second VHH comprising the amino acid sequence of SEQ ID NO: 8 or a homologous sequence thereof having at least 80% sequence identity yet retaining specific binding affinity to LAG-3.

4. The bispecific antibody or the antigen-binding portion thereof of claim 1, wherein from N terminal to C terminal, the first antigen binding domain is operably linked to a constant region, and the constant region is operably linked to the second antigen-binding domain, or vice versa.

5. The bispecific antibody or the antigen-binding portion thereof of claim 4, wherein the constant region is a human IgG constant region.

6. The bispecific antibody or the antigen-binding portion thereof of claim 5, wherein the human IgG constant region is a human IgG1 Fc region.

7. The bispecific antibody or the antigen-binding portion thereof of claim 6, wherein the human IgG1 Fc region comprises mutations of L234A and L235A, according to EU numbering.

8. (canceled)

9. The bispecific antibody or the antigen-binding portion thereof of claim 4, wherein the constant region is operably linked to at least one of the first antigen binding domain and the second antigen binding domain via a linker.

10. (canceled)

11. The bispecific antibody or the antigen-binding portion thereof of claim 9, wherein the linker comprises or consists of (G4S)n with n=1-10, optionally the linker consists of SEQ ID NO: 12.

12. (canceled)

13. The bispecific antibody or the antigen-binding portion thereof of claim 1, wherein the bispecific antibody or the antigen-binding portion thereof is a humanized antibody.

14. The bispecific antibody or the antigen-binding portion thereof of claim 1, wherein the full length of the antibody or the antigen-binding portion thereof comprises or consists of SEQ ID NO: 13.

15. An isolated nucleic acid molecule, comprising a nucleic acid sequence encoding the bispecific antibody or the antigen-binding portion thereof as defined in claim 1.

16. A vector comprising the nucleic acid molecule of claim 15.

17. A host cell comprising the vector of claim 16.

18. A pharmaceutical composition comprising the bispecific antibody or the antigen-binding portion thereof of claim 1 and a pharmaceutically acceptable carrier.

19. A method for producing the bispecific antibody or the antigen-binding portion thereof as defined in claim 1, comprising the steps of:
culturing a host cell comprising a nucleic acid sequence encoding the bispecific antibody or the antigen-binding portion thereof under suitable conditions; and
isolating the antibody or antigen-binding portion thereof from the host cell culture.

20. A method for modulating an immune response in a subject, comprising administering to the subject the bispecific antibody or the antigen-binding portion thereof as defined in claim 1 to the subject.

21. (canceled)

22. A method for preventing or treating a disease or condition in a subject, comprising administering an effective amount of the bispecific antibody or the antigen-binding portion thereof as defined in claim 1 to the subject, wherein the disease or condition is selected from a proliferative disorder, an immune disorder and an infection, and optionally the disease or condition is PD-L1 and/or LAG-3 related.

23. The method of claim 22, wherein the proliferative disorder is selected from colon cancer, lymphoma, lung cancer, liver cancer, cervical cancer, breast cancer, ovarian cancer, pancreatic cancer, melanoma, glioblastoma, prostate cancer, esophageal cancer, and gastric cancer, optionally a colon cancer.

24. The method of claim 22, wherein the infection is a chronic infection.

25-27. (canceled)

28. A kit for treating or diagnosing a proliferative disorders, an immune disorders or an infection, comprising a container comprising the bispecific antibody or the antigen-binding portion thereof as defined in claim 1.
